(19) United States
(12) Patent Application Publication
(10) Pub. No.: US 2019/0076434 A1
(43) Pub. Date: Mar. 14, 2019

(54) METHODS OF TREATING PERIPHERAL VASCULAR DISEASES, INCLUDING SYSTEMIC SCLEROSIS VASCULOPATHY

(71) Applicant: UNIVERSITY OF UTAH RESEARCH FOUNDATION, Salt Lake City, UT (US)

(72) Inventors: Anthony DONATO, Salt Lake City, UT (US); Tracy FRECH, Salt Lake City, UT (US)

(21) Appl. No.: 16/084,451

(22) PCT Filed: Apr. 29, 2016
(86) PCT No.: PCT/US16/30179

§ 371 Date: Sep. 12, 2018

Related U.S. Application Data
(60) Provisional application No. 62/317,086, filed on Apr. 1, 2016.

(51) Int. Cl. A61K 31/519; A61K 45/06

(52) U.S. Cl. CPC A61K 31/519; A61K 45/06

(57) ABSTRACT

Methods for treating diseases in which patients exhibit a reduced exercise capacity due to a decrease in their peripheral blood flow are provided, comprising administering an effective amount of tetrahydrobiopterin to the patient. The patient's resting blood flow is not affected by the administration, and smooth muscle dilating drugs may be administered to the patient concurrently with the tetrahydrobiopterin. Such diseases include chronic heart failure, peripheral vascular disease, diabetes and systemic sclerosis.

### CROSS REFERENCE TO RELATED APPLICATIONS

This application claims the benefit of U.S. Provisional Patent Application Ser. No. 62/317,086, filed Apr. 1, 2016, which is hereby incorporated by reference in its entirety for all of its teachings.

### STATEMENT REGARDING FEDERALLY FUNDED RESEARCH

This invention was made with government support under Grant Number VA ORD Merit CX001183-01A1 from the Department of Veteran's Affairs. The government has certain rights in the invention.

### FIELD OF INVENTION

The disclosure provided herein relates to methods for treating diseases in which patients exhibit a reduced exercise capacity due to a decrease in their peripheral blood flow, comprising administering an effective amount of tetrahydrobiopterin to a patient. Such diseases include systemic sclerosis, chronic heart failure, peripheral vascular disease, and diabetes.

### BACKGROUND

Systemic sclerosis (SSc; scleroderma) is a multi-organ systemic disease characterized by activation of immune cells, production of autoantibodies, vasculopathy, and fibrosis. Although SSc is heterogeneous in the extent of organ involvement and prognosis, it is accepted that all SSc cases have a progressive and usually devastating course. Presently there is no cure, no effective therapy to improve SSc, nor even a gold standard measurement of disease progression. The most common symptom in SSc is Raynaud's phenomenon that in its most severe form includes digital ulceration and gangrene. Indeed, early diagnostic criteria for SSc require the presence of Raynaud's phenomenon, capillary nailbed changes and/or SSc-specific antibodies. The goal of these early diagnostic criteria is to identify patients prior to end organ damage. However, the previously used “watchful watching” approach to therapy in Raynaud's patients is no longer acceptable, as a delay in SSc diagnosis results in higher morbidity and mortality.

Earlier SSc detection in Raynaud's patients is therefore needed to initiate treatments to improve clinical outcomes. In addition, existing therapies do not address the underlying physiologic dysfunction of SSc, but rather are often directed at symptom relief. There is no definitive data that current therapeutics for SSc actually slow progression of the severe end-stage manifestations of vasculopathy, such as pulmonary arterial hypertension (PAH) and digital ulcers (DU). While therapeutics are effective for scleroderma renal crisis (SRC), there is a lack of understanding of the etiology of this end-stage manifestation of SSc-related vasculopathy. These end organ manifestations contribute to substantial morbidity and mortality in patients, and limiting their progression is of critical importance. A better understanding of the initiating insult and natural progression of SSc is needed to better direct therapeutics with a goal of curing/treating the underlying disease. Importantly, vascular dysfunction is associated with all of these end organ manifestations of SSc.

SSc patients exhibit a reduced exercise capacity, together with a reduction in peripheral blood flow. Additional diseases which are also associated with a reduced flow of blood to the patient's periphery include chronic heart failure, peripheral vascular disease and diabetes.

### SUMMARY

The present invention relates to methods for treating systemic sclerosis, comprising administering an effective amount of tetrahydrobiopterin to a patient in need thereof. Methods for treating a disease which is associated with a patient exhibiting a reduced exercise capacity due to a decrease in their peripheral blood flow are also presented, comprising administering an effective amount of tetrahydrobiopterin to a patient in need thereof. These diseases include systemic sclerosis, chronic heart failure, peripheral vascular disease and diabetes.

Other aspects of the invention will become apparent by consideration of the detailed description and accompanying drawings.

### BRIEF DESCRIPTION OF THE DRAWINGS

The drawings below are supplied in order to facilitate understanding of the Description and Examples provided herein.

FIG. 1A and FIG. 1B are schematic illustrations of a coupled (FIG. 1A) and an uncoupled coupled (FIG. 1B) nitric oxide synthase (NOS) biochemical system.

FIG. 2A and FIG. 2B are graphs of brachial artery diameter (FIG. 2A) and forearm blood flow (FIG. 2B) in patients with scleroderma (open bars) compared to control patients (solid bars).

FIG. 3 shows graphs measuring flow-mediated dilation (FMD) and vascular reactivity in patients with scleroderma (open bars) compared to control patients (solid bars). Graph A (upper left) shows FMD as an absolute change in brachial artery diameter; graph B (upper right) shows FMD as a percentage relative to baseline diameter; graph C (lower left) shows reactive hyperemia as peak blood flow; and graph D shows reactive hyperemia as the area under the curve.

FIG. 4 shows graphs measuring flow-mediated dilation (FMD) and vascular reactivity in patients with scleroderma with digital ulcers (open bars) compared to scleroderma patients without digital ulcers (solid bars). Graph A (upper left) shows FMD as an absolute change in brachial artery diameter; graph B (upper right) shows FMD as a percentage relative to baseline diameter; graph C (lower left) shows reactive hyperemia as peak blood flow; and graph D shows reactive hyperemia as the area under the curve.

FIG. 5A-FIG. 5C shows the brachial artery flow-mediated dilation in centimeters (FIG. 5A) and as a percentage (FIG. 5B), and vascular reactivity (reactive hyperemia; FIG. 5C) in patients with scleroderma compared to control patients.

FIG. 6 is a graph correlating the percentage of patients with a digital ulcer with their flow-mediated dilation (FMD), indicating that high FMD is protective against digital ulcers.

FIG. 7A-FIG. 7C shows the improvement in flow-mediated dilation (FMD; FIG. 7A), reactive hyperemia as peak blood flow (FIG. 7B) and reactive hyperemia as the area under the curve (FIG. 7C) in patients with scleroderma after treatment with BH4.

FIGS. 8A and 8B are graphs showing the mean arterial pressure (MAP) for individuals (FIG. 8A) and as a group (FIG. 8B).

FIGS. 9A and 9B are graphs depicting the brachial artery (BA) flow-mediated dilation (FMD) responses between conditions.

FIGS. 10A and 10B are graphs showing the hemodynamic responses to 5-min cuff occlusion, in the brachial artery (BA) shear rate (FIG. 10A) and for the shear rate AUC (FIG. 10B) at peak dilation.

FIG. 11 shows graphs of the brachial artery (BA) nitroglycerin-mediated dilation (NMD) responses between conditions as a percentage (upper graph) and as an absolute value (lower graph).

FIG. 12 shows graphs of the handgrip force (as a percent of maximum) and mean arterial pressure (FIG. 12A), forearm blood flow (FIG. 12B), and forearm vascular conductance (FIG. 12C).

FIG. 13 shows a graph of the shear rate and vasodilation for healthy and SSc patients.

### DETAILED DESCRIPTION

Vasculopathy as a target for SSc therapeutics is important since vasculopathy precedes fibrosis. An integrative approach to study vasculopathy in SSc is needed, specifically, a better definition of the pathophysiology of vasculopathy, validation of the use of non-invasive endothelial function testing, testing the efficacy of BH4 supplementation and exploring molecular mechanisms related to oxidative stress in SSc. This approach can dramatically improve both patient care and clinical outcomes in SSc.

The pathogenesis of Raynaud's phenomenon is not fully understood, but is known to involve vascular dysfunction. Dysfunction in the vascular microenvironment may be a critical factor in the transition from Raynaud's to SSc. Vascular dysfunction in Raynaud's phenomenon results from abnormalities in the functional capacity of the cells within the vascular microenvironment including endothelial cells, smooth muscle cells and nerve terminals. Raynaud's phenomenon is associated with impaired tissue perfusion that leads to tissue hypoxia and endothelial cell damage. Damage to endothelial cells, and resulting endothelial dysfunction, is linked to many of the end-stage vascular abnormalities of SSc. The measurement of endothelial function holds promise as a novel method to assess disease progression and therapeutic efficacy in SSc. In addition, pharmacologic compounds that target the endothelium represent a novel therapeutic approach with great promise to reduce SSc-related tissue hypoxia and end organ damage, as well as potentially impact underlying disease progression.

SSc-related vasculopathy and the endothelium. The endothelium, a single layer of cells lining the lumen of the vessel wall, is an important regulator of vascular function and homeostasis. Damage to the endothelium resulting from a sustained exposure to this hypoxic environment can result in apoptosis (i.e., cell death) that, when coupled to insufficient repair, results in the pathognomonic end-stage vascular abnormalities of SSc. SSc is associated with impaired peripheral vascular endothelial function characterized by increased vascular permeability, immune cell adhesion and infiltration, blunted angiogenic capacity and reduced ability to dilate, all of which are involved with dictating the rate of development of vasculopathy. Proper functioning of the endothelium and maintenance of a disease resistant artery depends on production of the critical endothelial derived molecule, nitric oxide (NO). Vascular NO bioavailability is evidenced by an intact, robust endothelium-dependent dilation and is, in part, responsible for mediating the angiogenic capacity, peripheral permeability, and anti-inflammatory properties of a healthy vascular endothelium. Thus, maintaining appropriate bioavailability of NO in the vascular endothelium preserves vascular endothelial function and health, and subsequently may prevent complications associated with vasculopathy in SSc.

Functions of the endothelium: Endothelium Dependent Dilation (EDD). NO is the primary vasodilatory molecule released from the vascular endothelium in response to stimulation by agonists, e.g. insulin, acetylcholine, or changes in shear stress. In the vascular endothelium, NO is produced by the enzyme endothelial NO synthase (eNOS) and subsequently released from endothelial cells. NO released from the endothelium diffuses to the vascular smooth muscle where it causes relaxation and increased blood vessel diameter. Endothelial dysfunction, characterized by reduced NO and impaired vasodilator capacity, results in diminished peripheral tissue blood flow.

Angiogenesis. Angiogenesis, i.e., new artery growth, is required for the appropriate expansion of the tissue during growth or in times of sustained or frequent tissue hypoxia. In healthy tissue, hypoxic stress stimulates the pro-angiogenic transcription factor hypoxia inducible factor 1α (HIF1α), leading to increased angiogenic factors, such as vascular endothelial growth factor (VEGF) and subsequent angiogenesis. In contrast, with hypoxia in SSc HIF1α60 and VEGF are increased, but these do not lead to angiogenesis due to enhanced angiogenic inhibitors such as endostatin. Endostatin is associated with reduced eNOS activation, reduced NO production and apoptosis. Furthermore, the angiogenic actions of VEGF signaling are dependent on a functional vascular endothelium and presence of NO.

Leukocyte Adhesion/Infiltration. Increased infiltration of immune cells leads to the peripheral tissue inflammation that has been directly implicated in the etiology of SSc. The increased immune cells in peripheral tissues of patients with SSc, likely result from greater adhesion of immune cells to the vascular endothelium. While a healthy endothelium can serve as a barrier to the movement of immune cells from the circulation into tissues, the unhealthy endothelium in SSc can augment the inflammatory process, thereby stimulating greater infiltration of immune cells into the peripheral tissues.

Thus, dysfunction of the vascular endothelium and reductions in NO bioavailability with SSc likely contribute to tissue hypoxia via diminished blood flow and an inadequate angiogenic response to tissue hypoxia. In addition, the promotion of tissue inflammation and associated immune cell infiltration may further reduce NO bioavailability and endothelial function by increasing vascular oxidative stress.

Oxidative Stress, NO and endothelial function. Oxidative stress, defined as an excess production of free radicals relative to antioxidant defenses, has been documented in SSc. Serum and urinary markers of systemic oxidative stress are greater in SSc compared with healthy age matched controls. The functional consequences of oxidative stress are widespread, but the vascular endothelium is particularly vulnerable to oxidative damage from reactive oxygen species (ROS). NO produced by the endothelium reacts with superoxide to form the ROS peroxynitrite (ONOO—), resulting in reduced NO available to signal vasodilation. ROS production, including superoxide and ONOO— formation, is increased in the circulation and skin of patients with SSc. Thus, oxidative stress is implicated as a major contributor to the reduced NO bioavailability and endothelial dysfunction, and leads to the deleterious endothelial phenotype characterized by enhanced permeability, reduced peripheral blood flow, increased immune cell adhesion and infiltration and increased local arterial inflammation.

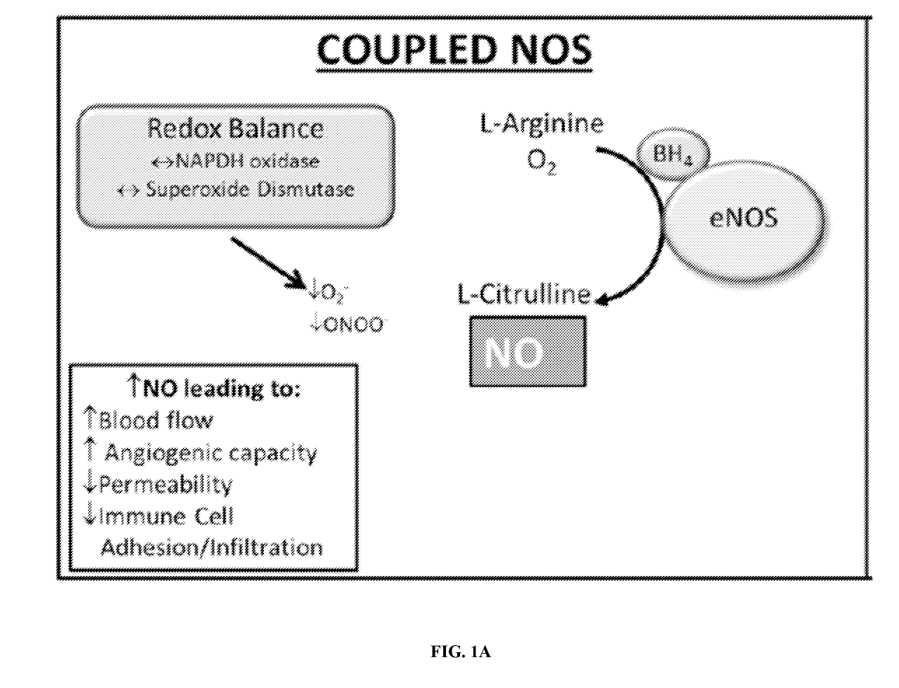

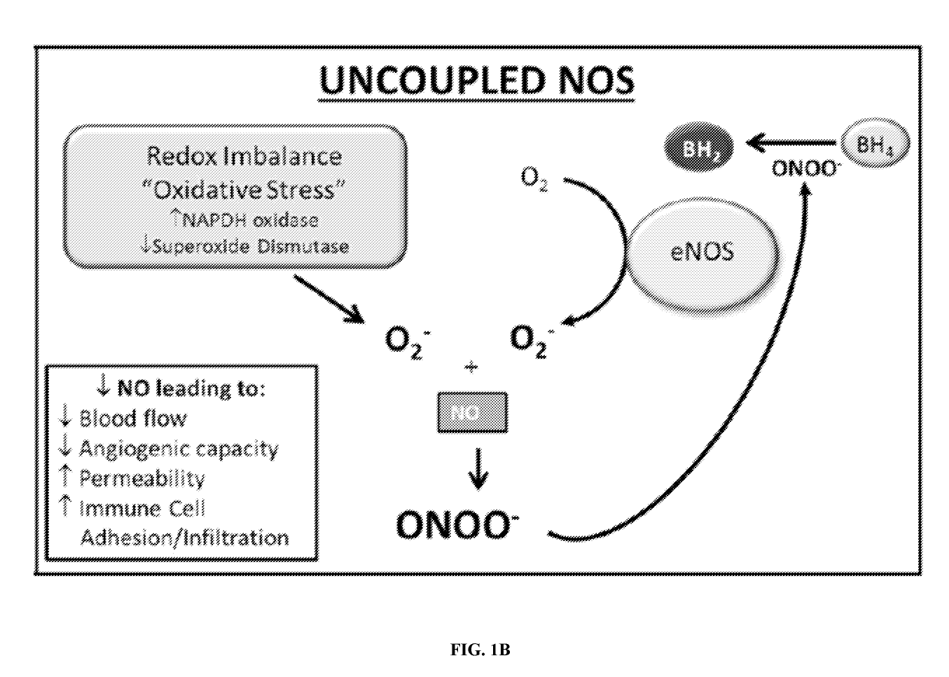

The role of tetrahydrobiopterin (BH4) in NO bioavailability. BH4 is an essential cofactor for eNOS and maintains NO bioavailability in the vascular endothelium, as shown schematically in FIG. 1A. When the concentration of BH4 is insufficient in the endothelial cell, eNOS becomes “uncoupled,” as shown in FIG. 1B. Uncoupled eNOS no longer produces NO, but rather produces superoxide. The increased superoxide can lead to peroxynitrite (ONOO—) formation which, in turn, oxidizes BH4 to its inactive form (BH2), leading to further eNOS uncoupling, greater superoxide formation, and reduced NO production and bioavailability in a “vicious cycle.”

Restoring BH4 concentrations can stop this vicious cycle, resulting in reduced superoxide production, reduced ONOO— formation and oxidation of BH4, increased NO production and bioavailability, and improved endothelial function. Thus, maintaining sufficient BH4 concentrations in the vascular endothelium can maintain vascular endothelial health (as reflected by enhanced EDD) and prevent vascular disease.

BH4 Supplementation. Acute infusion of a high dose of BH4 improves endothelial function measured via peripheral vasodilation in patients with hypercholesterolemia, diabetes, hypertension, chronic heart failure, and in smokers. Acute oral BH4 administration ameliorates these phenotypes in patients with cardiovascular disease as well as healthy older adults. BH4 supplementation for 4 days at a therapeutic dose (5 mg/kg/day) improves EDD in patients with hypercholesterolemia however, to date the efficacy of BH4 supplementation in restoring vascular and/or endothelial function in patients with SSc has not been assessed. Initial studies indicated that acute administration of oral high-dose BH4 improves endothelial function in patients with DU, a SSc-related end-stage “complicated” vasculopathy.

Previous investigations using BH4 to treat cardiovascular disease (CVD) have been unsuccessful, and the enzymatic target identified herein for treating SSc is different than those historically targeted for treating CVD.

Notably, the vasculature is a complex tissue made of multiple cell types (endothelial, vascular smooth muscle, fibroblasts, immune cells, adipocytes etc.). The current standard of care for treating SSc (and CVD in general) targets the vascular smooth muscle cells to relax the smooth muscle and produce vasodilation, thereby increasing blood flow. These drugs affect the endothelin-A (ET-A) receptor and calcium channels on the vascular smooth muscle. BH4, in contrast, is an essential cofactor for an endothelial enzyme, endothelial nitric oxide synthase, which produces nitric oxide. Nitric oxide is a regulator of endothelial health and critical for appropriate function of the endothelium. BH4 is not in the same class as smooth muscle dilating drugs, and targets a different cell type and enzymatic pathway to improve endothelial function. BH4 targets the vascular endothelium to improve vasculopathy associated with SSc.

Furthermore, BH4 has failed to show efficacy in treating CVD in the clinic. SSc is a rare and lethal disease and is not treatable with existing CVD drugs, including statins, beta blockers, and acetylcholinesterase inhibitors. Several clinical trials testing BH4 for treating uncontrolled hypertension and atherosclerosis have failed. Thus, there are no current therapeutic regimens targeted at improving endothelial function in patients with SSc.

The methods disclosed herein demonstrate a method of treating systemic sclerosis comprising administering an effective amount of tetrahydrobiopterin to a patient in need thereof. The methods also demonstrate a method of treating a disease which results in, causes, or is associated with a reduced exercise capacity due to a decrease in a patient's peripheral blood flow, comprising administering an effective amount of tetrahydrobiopterin to a patient in need thereof. Such diseases include systemic sclerosis, chronic heart failure, peripheral vascular disease and diabetes.

In some embodiments, the patient's mean arterial blood pressure is not substantially affected by the administration, such as it does not change by more than 10%. In certain embodiments, the patient's brachial artery shear rate is not substantially affected by the administration, such as it does not change by more than 10%. The method can further include administration of at least one smooth muscle dilating drug in addition to the tetrahydrobiopterin, or administration of at least one drug which is used to treat pulmonary hypertension and which causes constriction of the pulmonary blood vessels.

The methods disclosed herein include treating systemic sclerosis by administering an effective amount of tetrahydrobiopterin orally, which can occur once a day or more than once a day. The dosage of tetrahydrobiopterin may range from about 0.5 mg/kg to about 50 mg/kg, or from about 1 mg/kg to about 25 mg/kg, or from about 1 mg/kg to about 15 mg/kg, or be about 10 mg/kg.

Upon treatment with tetrahydrobiopterin, the patient may exhibit at least one of the following: the patient having an average brachial artery dilation to flow of at least about 0.20 mm; the patient having an increase in their average brachial artery dilation to flow of at least about 30% as compared to the average prior to treatment; the patient having an increase in their average brachial artery flow mediated diameter of at least about 0.05 mm compared to their average diameter prior to treatment; the patient having an increase in their average brachial artery flow mediated diameter of at least about 2% compared to their average diameter prior to treatment; the patient showing a decrease in their risk of getting a digital ulcer as compared to a patient administered a placebo; the patient showing a decrease in their risk of getting a digital ulcer from about 45% to about 15% as compared to a patient administered a placebo; the patient having an increase in their average forearm blood flow of at least about 20% as compared to their average forearm blood flow prior to treatment; or the patient having an increase in their average forearm blood flow of at least about 30 mL/min as compared to their average forearm blood flow prior to treatment.

Systemic sclerosis (SSc) is a multi-organ systemic disease that is associated with an impaired ability of the endothelium to produce nitric oxide (NO) and induce vasodilation. Tetrahydrobiopterin (BH4) is an essential cofactor for endothelial NO synthase (eNOS) and is critical for maintaining a healthy vascular endothelium. Initial studies indicated that SSc is characterized by abnormal vascular endothelial cells, impaired vascular endothelial function and impaired vascular reactivity. These results also demonstrated that measures of vascular endothelial function can provide important insight about SSc disease progression and prognosis, and evidence that BH4 can improve vascular function in patients with SSc.

An additional study confirmed that acute oral BH4 supplementation restores endothelial function in patients with SSc. Using a double-blind, randomized, crossover design, brachial artery (BA) flow-mediated dilation (FMD) was determined in 11 patients with SSc (61±3 years) five hours after oral BH4 (10 mg/kg) or placebo supplementation. Following acute BH4 supplementation, FMD was significantly higher compared to placebo (+48%, P<0.05). Specifically, improvements in FMD of at least 20% were present in nine of the eleven subjects in the BH4 condition compared to placebo. No differences were observed in the magnitude of post-cuff release shear rate area under the curve (AUC) between conditions (P>0.05), indicating that improvements in FMD in the BH4 condition were achieved independent of the post-cuff release shear rate AUC. These results indicated that acute BH4 supplementation improves FMD despite similar post-cuff release shear rate response and in the absence of improvements in smooth muscle vasoreactivity compared to placebo. These findings support the idea that improvements in FMD following acute BH4 supplementation are achieved by a restoration of vascular endothelial function in patients with SSc.

Before any embodiments of the invention are explained in detail, it is to be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof, as well as additional items.

It also should be understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

It should be understood that, as used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

Reference throughout this specification to “one embodiment,” “an embodiment,” or similar language means that a particular feature, structure, or characteristic described in connection with the embodiment is included in at least one embodiment of the invention provided herein. Thus, appearances of the phrases “in one embodiment,” “in an embodiment,” and similar language throughout this specification may, but do not necessarily, all refer to the same embodiment.

Furthermore, the described features, structures, or characteristics of the methods provided herein may be combined in any suitable manner in one or more embodiments. In the following description, numerous specific details are provided, to provide a thorough understanding of embodiments. One skilled in the relevant art will recognize, however, that the embodiments may be practiced without one or more of the specific details, or with other methods, components, materials, and so forth. In other instances, well-known structures, materials, or operations are not shown or described in detail to avoid obscuring aspects of the embodiments.

### EXAMPLES

Endothelial cells are abnormal in patients with SSc. The inventors have previously established that patients with SSc have greater vascular leakage and abnormal endothelial cells compared with healthy control subjects. Specifically, using whole-field digital microscopy, patients with SSc were found to have greater interstitial edema, a marker of vascular permeability, (31.0%±9.6% vs 17.6%±3.3% in controls; p=0.009) and fibrosis (75.6%±5.7% vs 66.1%±9.8% in controls; p=0.02). Patients with SSc also had lower CD34 staining compared with healthy controls (0.32%±0.22% vs 1.31%±0.34%; p<0.0001), indicating a reduced potential for vascular repair.

In addition, perivascular and interstitial infiltrate of mast cells was present in all SSc specimens. Using transmission electron microscopy, it was determined that all SSc specimens had endothelial swelling. These findings indicate that SSc is characterized by dysfunctional endothelial cells that lead to increased vascular permeability. Endothelium Dependent Dilation (EDD) is also impaired in patients with SSc.

Exemplary embodiments of the present disclosure are provided in the following examples. The examples are presented to illustrate the inventions disclosed herein and to assist one of ordinary skill in making and using the same. These are examples and not intended in any way to otherwise limit the scope of the inventions disclosed herein.

### Example 1

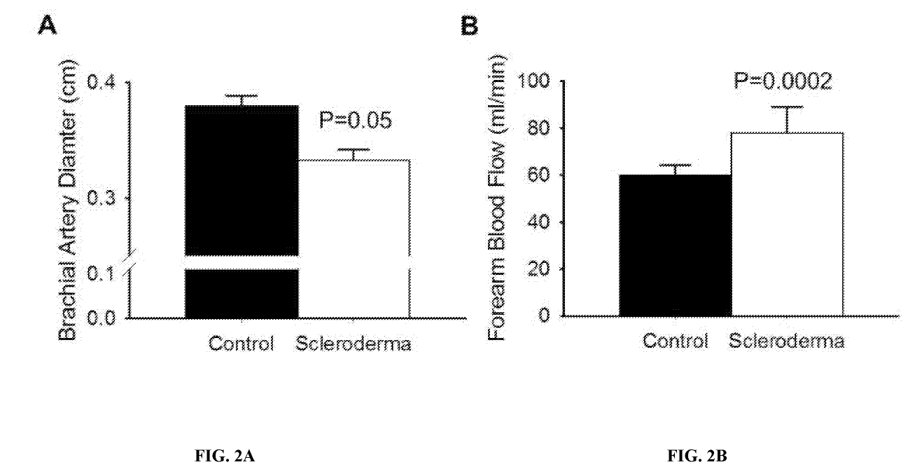

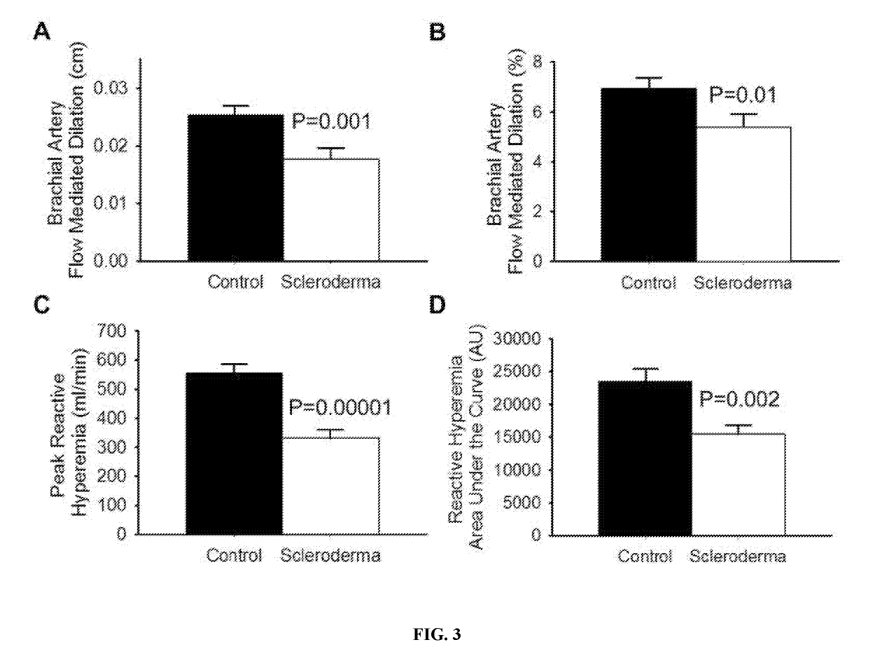

In an initial study, 38 patients with SSc and 51 healthy age- and BMI-matched control subjects were enrolled. In the SSc population, there was evidence of end organ damage due to SSc-vasculopathy in 29 patients (DU=14, PAH [determined by right heart catherization]=14, and/or SRC=1). Patients with SSc had a lower baseline brachial artery diameter and greater resting forearm blood flow compared with healthy controls, as shown in the graphs of FIG. 2.

FIG. 2A is a graph comparing the brachial artery diameter of control (solid bar) and SSc (open bar) patients. FIG. 2B is a graph comparing the forearm blood flow of control (solid bar) and SSc (open bar) patients. The data shows that brachial artery diameter is lower, but baseline blood flow is higher, in patients with scleroderma compared to control subjects. The data were assessed by Doppler ultrasound at rest, and data are mean±SEM, with P values denoting the difference from control.

It appeared that this augmented forearm blood flow is the result of peripheral tissue inflammation as well as the medications used to treat SSc. This is evident in the graphs shown in FIG. 3, which measure flow-mediated dilation (FMD) and vascular reactivity in patients with scleroderma (open bars) compared to control patients (solid bars). Data are mean±SEM, with P values denoting the difference from control.

Vascular reactivity, measured as the reactive hyperemia response, was reduced by ˜40% in patients with SSc compared with controls (FIG. 3, graphs C and D). In addition, endothelial function measured by brachial artery flow-mediated dilation (FMD) was impaired by ˜30% in patients with SSc compared with controls (FIG. 3, graphs A and B). Lower reactive hyperemia in patients with SSc resulted in lower shear stress on the vascular wall (P=0.03). However, when FMD is normalized for brachial artery shear stress, vasodilation in the SSc groups remained significantly impaired compared with healthy controls (P<0.05, data not shown). Thus, SSc is characterized by profound vascular dysfunction

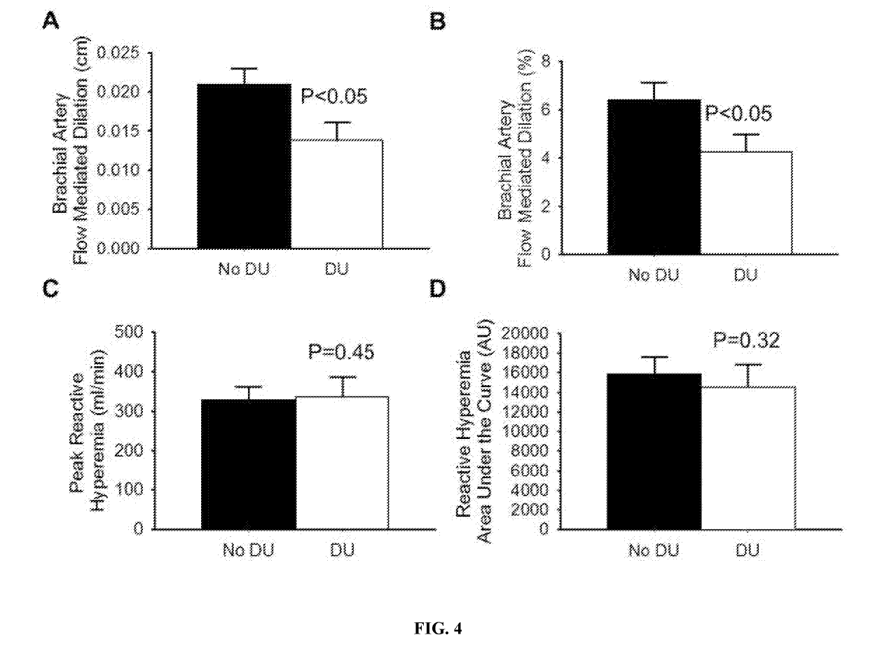

Preserved endothelial function may be protective against end-stage SSc vasculopathy. The SSc group was divided into patients with and without digital ulcers (DU) and/or pulmonary arterial hypertension (PAH). SSc patients did not differ significantly by disease duration, antibody status, or modified Rodnan skin scores. Within scleroderma patients, the presence of digital ulcers was associated with greater impairments in endothelial function, as shown in graphs of FIG. 4. Endothelial function was assessed by comparing FMD and forearm blood flow, assessed by reactive hyperemia, in patients with scleroderma with DU (open bars) compared to patients with scleroderma with no DU (solid bars). Data are mean±SEM, with P values denoting the difference from patients without DU.

Patients with DU had impaired endothelial function, as evidenced by ˜20% lower FMD, compared with patients without this end-organ complication (FIG. 4, graphs A and B). However, reactive hyperemia was similar between patients with SSc with and without DU (FIG. 4, graphs C and D). Likewise, shear stress as well as baseline forearm blood flow and brachial diameter were similar between groups (not shown, all P>0.05). SSc patients with and without PAH had significant differences FMD maximum diameter (p=0.02) and shear stress (p=0.04), suggesting patients with PAH not only had smaller brachial arteries, but also perhaps less of an ability to dilate those small arteries. FMD measurements were not affected by use of calcium channel blockers, phosphodiesterase inhibitors, endothelin receptor anatagonists, and/or prostacyclin analogs. These results indicate that impaired endothelial function per se may be associated with clinical complications of end-stage vasculopathy in SSc. Therefore, measures of endothelial function may be important in determining the stage of SSc progression.

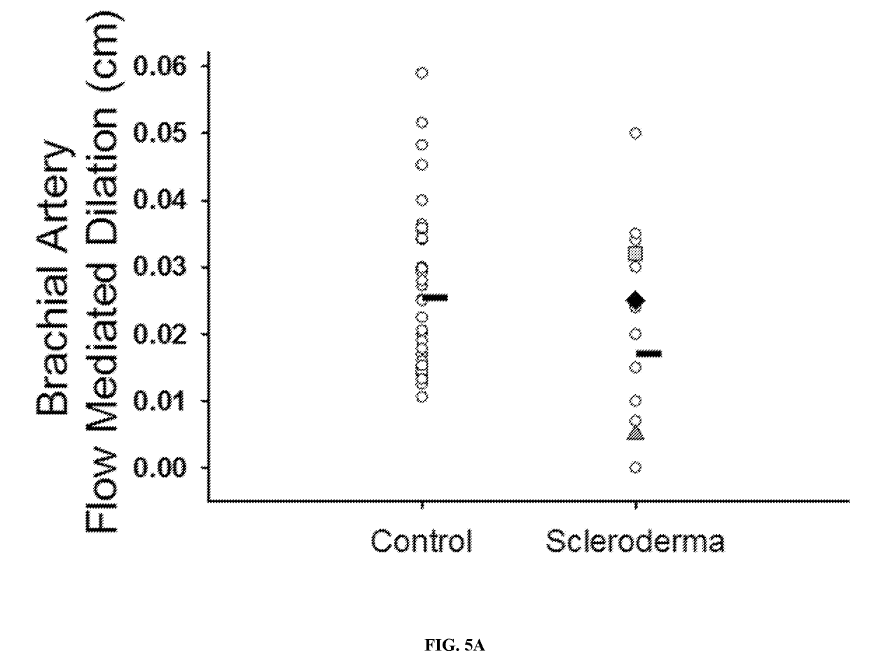

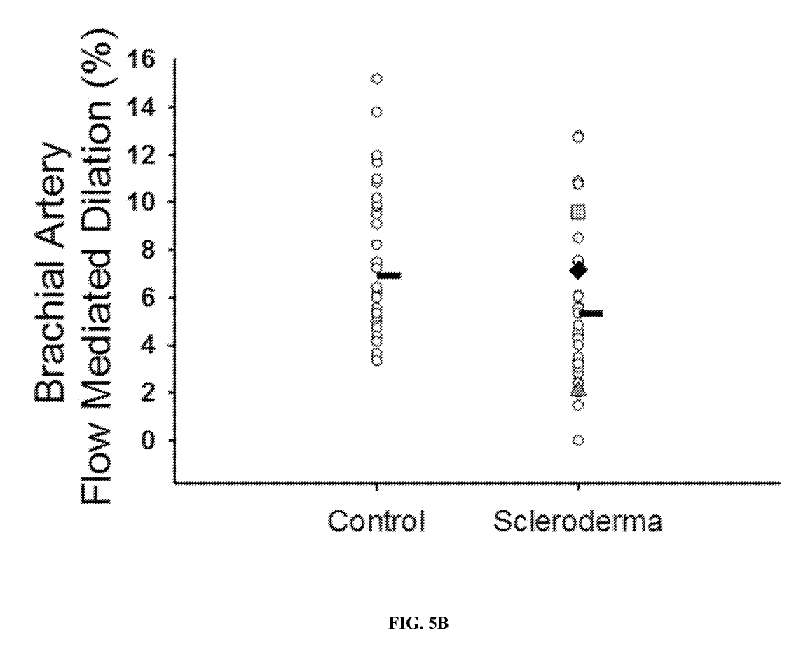

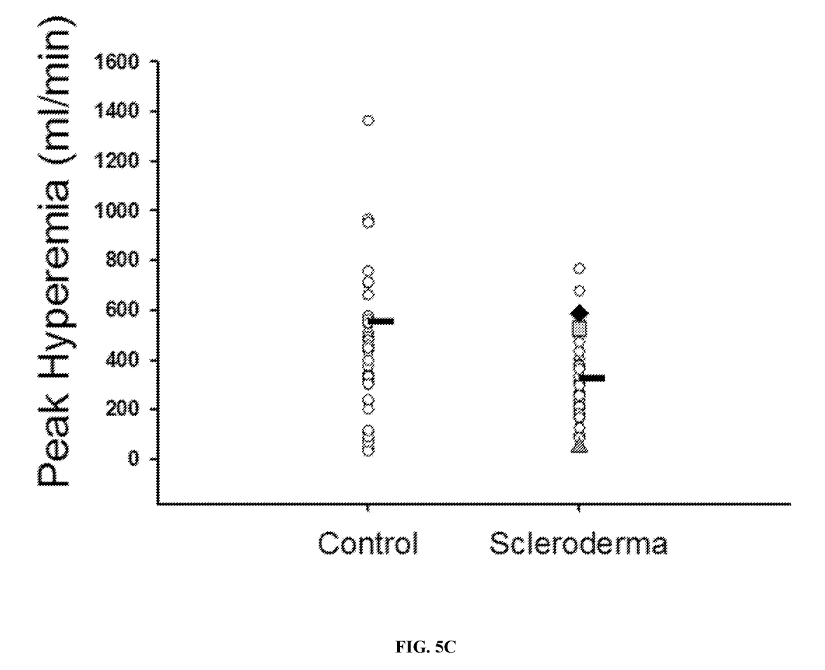

Low FMD in patients with SSc may be predictive of increased risk for development of end-stage or “complicated” vasculopathy. For 3 subjects who did not present with DU at baseline testing, a 4 month follow-up clinical assessment was performed. Analysis of the vascular function of these subjects is shown in FIG. 5, with the triangle showing data from a patient without a DU at baseline, but who developed DU by follow-up. The square and diamond indicate patients without DU at baseline and follow-up.

At follow-up, the subject with below average vascular function at baseline (triangle) had subsequently developed a DU, and in contrast, the two subjects with above or average vascular function at baseline (square and diamond) did not develop DU. These initial findings suggested that measures of endothelial function and vascular reactivity may provide important clinical insight into prognosis for patients with SSc. None of the study subjects developed PAH or SRC during 4 month testing period.

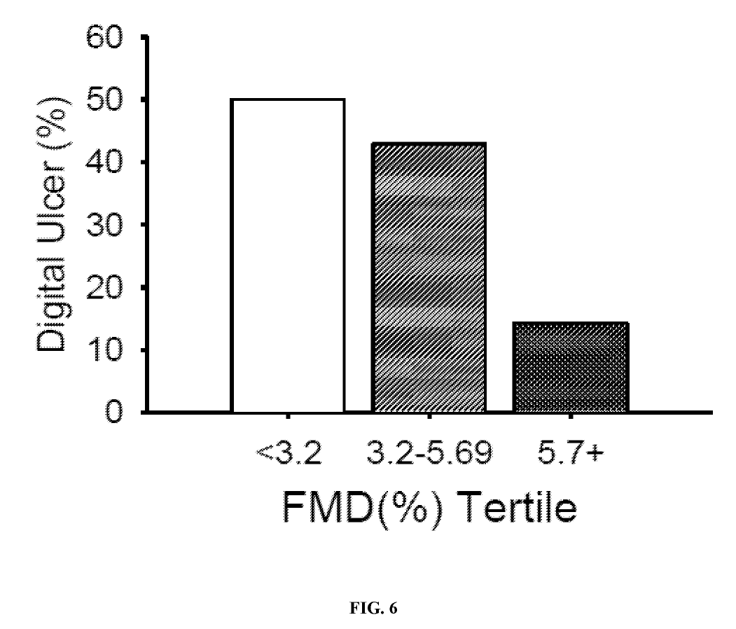

To explore the predictive value of FMD for DU, FMD (%) data for all patients with SSc was divided into tertiles and the odds ratio of having a digital ulcer was determined. High FMD was protective from DU, with 40-50% of patients in the lower two FMD tertiles presenting with DU compared to only 14% of those in the upper FMD tertile, as shown in FIG. 6. The calculated odds ratio suggests that those in the lower two FMD tertiles (absolute FMD <0.0239 cm) have ˜5 times greater risk of developing DU compared to those patients with the highest FMDs (absolute FMD≥0.024 cm). The P value for the trend=0.024, and N=14 per tertile.

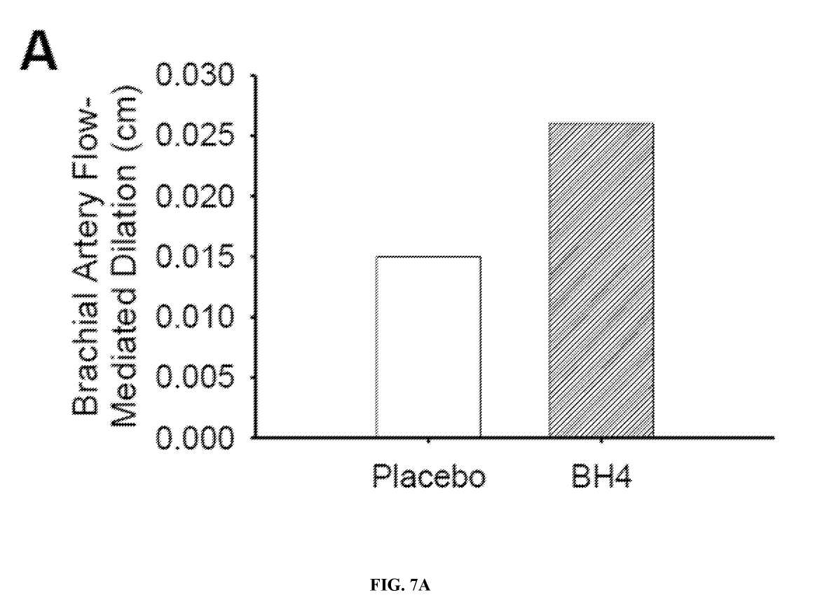

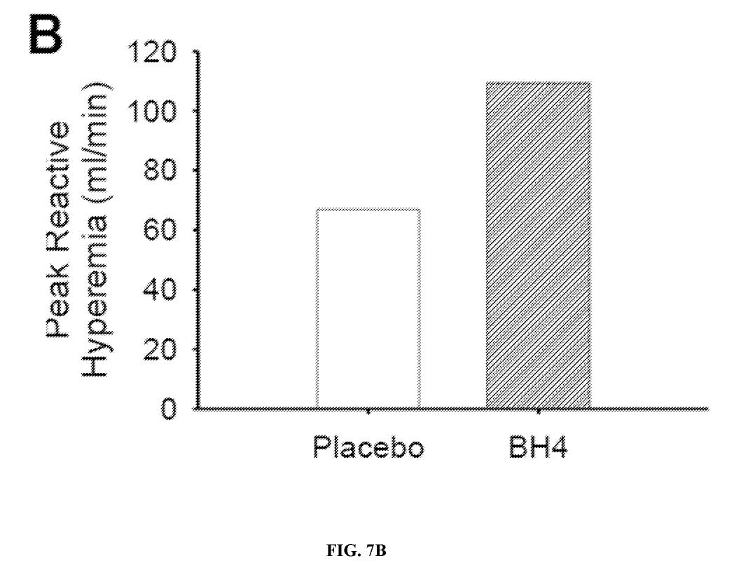

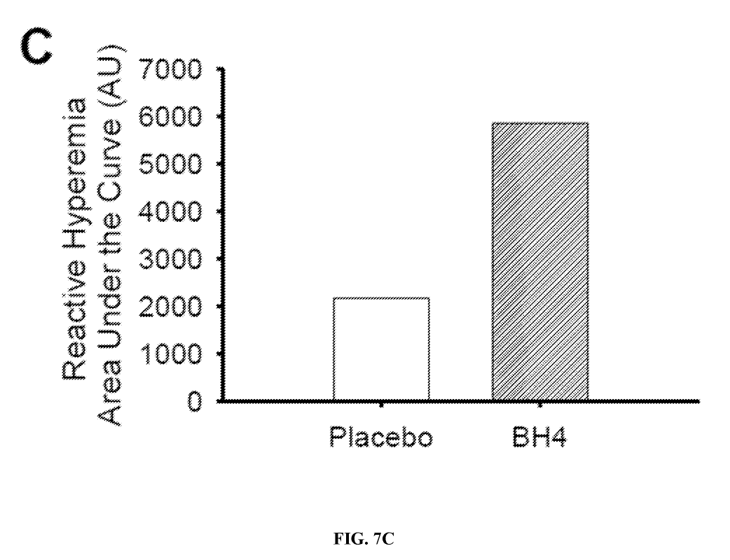

Tetrahydrobiopterin (BH4) treatment may improve endothelial function in patients with SSc. The effect of BH4 treatment on vascular function was assessed in the subject whose baseline vascular measures are shown above as the red circle in FIGS. 5A-5C. Vascular measures were assessed on this subject after treatment with placebo and BH4 administration. BH4 treatment resulted in a modest effect on resting blood flow in the forearm (˜20% increase) and leg (34% increase)(data not shown); and a robust effect on FMD (73% increase, FIG. 7A), peak hyperemia (63% increase, FIG. 7B) and the area under the curve for hyperemia (˜160% increase, FIG. 7C) compared to placebo.

Interestingly, BH4 did not have a marked influence on blood pressure (126/63 vs. 125/77, placebo vs BH4). Taken together with the odds ratios calculated above, the magnitude of increase in FMD after BH4 corresponds to this subject moving from the lowest FMD (high risk) tertile to the highest FMD (low risk) tertile. Although only one patient has been tested before and after BH4 and placebo administration, the large improvements in FMD and hyperemia observed after BH4 in this subject are promising.

This initial study indicated that SSc is characterized by abnormal vascular endothelial cells, impaired vascular endothelial function and impaired vascular reactivity. These results also demonstrate that measures of vascular endothelial function can provide important insight about SSc disease progression and prognosis, and evidence that BH4 can improve vascular function in patients with SSc.

These studies provided the basis for a non-invasive technique to evaluate vasculopathy in SSc and an evaluation of the efficacy of BH4 in ameliorating vascular dysfunction in patients with SSc. Further, the link between oxidative stress and vascular endothelial dysfunction in SSc can be analyzed to provide further mechanistic insight, and dramatically improve both patient care and clinical outcomes in SSc.

### Example 2

Despite evidence that endothelial cell dysfunction may contribute to vasculopathy in patients with SSc, the current standard of care for treatment of SSc is the prescription of smooth muscle vasodilators (i.e., channel blockers, phosphodiesterase inhibitors, endothelin receptor antagonists, and/or prostacyclin analogs), not endothelium targeted drugs. In light of this, the endothelium represents a novel and robust target for drug treatment in SSc, as restoration of endothelial function in patients with SSc may also delay disease progression and reduce risk of future vasculopathy. This study investigated whether acute oral BH4 supplementation improved vascular endothelial function, as determined by brachial artery (BA) flow-mediated dilation (FMD).

Methods: Subjects. Eleven subjects with SSc (3 men and 8 women) were recruited from the University of Utah SSc clinic to participate in the current study. All subjects had either a diagnosis of SSc as accepted by the American College of Rheumatology or early SSc as described by Leroy and Medsger. All subjects gave their written informed consent before participation. Protocol approval and written informed consent were obtained, according to the University of Utah and Salt Lake City Veterans Affairs Medical Center (VAMC) Institutional Review Board, in accordance with the principles outlined in the Declaration of Helsinki. All data collection took place in the Utah Vascular Research Laboratory at the Salt Lake City VAMC Geriatric Research, Education, and Clinical Center.

Experimental Design. A controlled, randomized, double-blind, crossover experimental design with two conditions, BH4 and placebo, was used, with a washout period of ≥5 days between conditions. Subjects consumed a standardized breakfast and were administered oral BH4 (10 mg/kg) or placebo five hours prior arrival at the laboratory. Measurements were taken at the same time of day to eliminate any diurnal effects and after having abstained from food (not including the standardized breakfast), alcohol, caffeine, cardiovascular-acting medications, and exercise for ≥12 hours. In premenopausal women, measurements were performed during the early follicular phase of the menstrual cycle.

Blood Pressure. Seated BA blood pressure measurements were made with a semi-automated BP device (Tango+, SunTech, Morrisville, N.C.) in triplicate after 5 min in the upright seated position with the arm at heart level and under quiet, comfortable, ambient (˜22° C.) laboratory conditions. Mean arterial pressure (MAP) was calculated using the equation: ⅓ systolic blood pressure+⅔ diastolic blood pressure.

Flow-Mediated Dilation (FMD). The FMD procedures were performed in accordance with standard guidelines. Briefly, a blood pressure cuff was place on the right arm, distal to the ultrasound Doppler probe on the BA. Simultaneous measurements of BA blood velocity and vessel diameter were performed using a linear array transducer operating in duplex mode, with imaging frequency of 14 MHz and Doppler frequency of 5 MHz (Logic 7, GE Medical Systems, Milwaukee, Wis.). All measurements were obtained with the probe appropriately positioned to maintain an insonation angle of ≤60°. The sample volume was maximized according to vessel size and was centered within the vessel on the basis of real-time ultrasound visualization. The BA was insonated approximately midway between the antecubital and axillary regions, and measurements of diameter and blood velocity (Vmean) were obtained continuously at rest and for 2 minutes after cuff deflation. End-diastolic, ECG R-wave-gated images were collected via video output from the Logic 7 for off-line analysis of BA vasodilation using automated edge-detection software (Medical Imaging Application, Coralville, Iowa). Heart rate was monitored from a standard 3-lead ECG. In a subset of patients (n=7), bedside FMD measurements were performed with patients when on their normal medication routine at the University of Utah Rheumatology Clinic.

Nitroglycerin-mediated dilation (NMD). When not contraindicated due to medication usage or disease progression, NMD was assessed in a subset of subjects (n=4). At least 60 min after FMD assessment, measurement of BA vessel diameter was performed at baseline and during the 5 min after sublingual nitroglycerin (0.8 mg) administration.

Analyses. FMD was quantified as the maximal change in BA diameter after cuff release. NMD was quantified as the maximal change in BA diameter after sublingual nitroglycerin supplementation. FMD and NMD are expressed in both absolute units (i.e., Δ mm) and as a percentage increase (%Δ) above baseline. Shear rate was calculated according to the equation: shear rate (s−1)=(Vmean·8)/vessel diameter. Blood flow was calculated as per the equation: blood flow (mL·min)=(blood velocity×π)×(vessel diameter/2)2×(60). Cumulative shear rate cumulative area under the curve at the time of peak BA vasodilation was integrated with the trapezoidal rule and calculated as per the following equation: τ(yi[x(i+1)−xi]+(1/2)[y(i+1)−yi][x(i+1)−xi]). Forearm vascular conductance was calculated according to the equation: MAP/BA blood flow. Forearm vascular resistance was calculated according to the equation: BA blood flow/MAP.

Statistics. Statistics were performed using SPSS software (IBM, Chicago, Ill.). Paired, one-tailed t-tests were used to identify significant changes in measured variables between placebo and BH4 conditions. Statistical significance was set at P<0.05 for all analyses. Unless indicated otherwise, data are presented as Means±SEM.

Results. Subject characteristics are presented in Table 1. There were no observations of, nor was there sufficient statistical power to identify, any effects of disease duration, end-stage vasculopathy, or antibody presence on the response of cardiovascular between the BH4 and placebo condition.

TABLE 1
Selected Subject Characteristics.

| Variables | |
|---|---|
| Women:men, n | 8:3 |
| Age, years | 61 ± 11 |
| Height, cm | 170 ± 9 |
| Weight, kg | 71.3 ± 13 |
| BMI, kg/m² | 24.8 ± 4.4 |
| Medications, % | |
| Calcium channel blockers | 73 |
| Angiotensin II receptor antagonists | 0 |
| ACE inhibitors | 0 |
| Endothelin receptor antagonists | 9 |
| Phosphodiesterase inhibitors | 9 |
| Immunosuppression | 36 |
| Prostacyclin analog | 0 |
| SSc duration, years | 7.6 ± 8.9 |
| End-stage vasculopathy, % | |
| Digital ulcers | 18 |
| SSc renal crisis | 9 |
| Pulmonary arterial hypertension | 18 |
| Antibody presence, % | |
| Centromere | 45 |
| RNA polymerase III | 9 |
| SCL70 | 22 |
| Fibrillin | 20 |
| Th/To | 0 |
| RNP | 18 |

Values are Means ± SD.

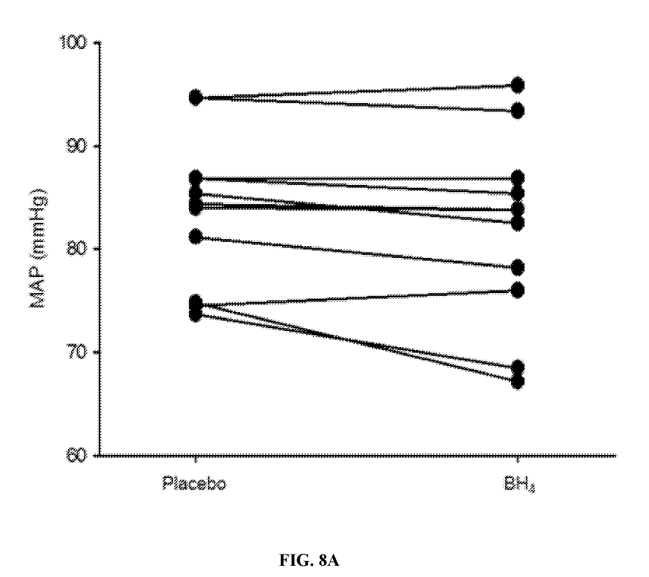

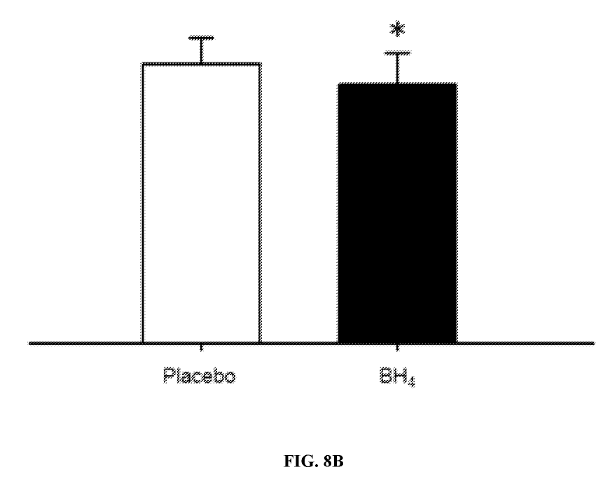

There were no differences in casual seated systolic (placebo: 112±4 vs. BH4: 109±5 mmHg, P>0.05) and diastolic (placebo: 70±2 vs. BH4: 68±2 mmHg, P>0.05) blood pressure between conditions. Despite no statistical differences in systolic or diastolic blood pressure between conditions, MAP was slightly, but significantly lower (˜−2 mmHg) in the BH4 condition, as compared to placebo, for individuals (shown in FIG. 8A) and as a group (shown in FIG. 8B). MAP was lower after treatment with BH4 compared to placebo; *P<0.05 vs. placebo; values are Means±SEM.

As depicted in Table 2, there were no differences in selected cardiovascular variables at baseline between conditions (P>0.05).

TABLE 2
Changes in Selected Cardiovascular Variables.

| Variables | Placebo | BH₄ |
|---|---|---|
| Heart rate, bpm | 66 ± 3 | 66 ± 3 |
| BA diameter, mm | 3.7 ± 0.2 | 3.7 ± 0.2 |
| BA blood flow, ml/min | 33 ± 7 | 28 ± 4 |
| Vascular resistance, U | 3.4 ± 0.5 | 3.4 ± 0.4 |
| Vascular conductance, U | 0.38 ± 0.07 | 0.34 ± 0.04 |

Values are Means ± SEM.
There were no significant differences between conditions.

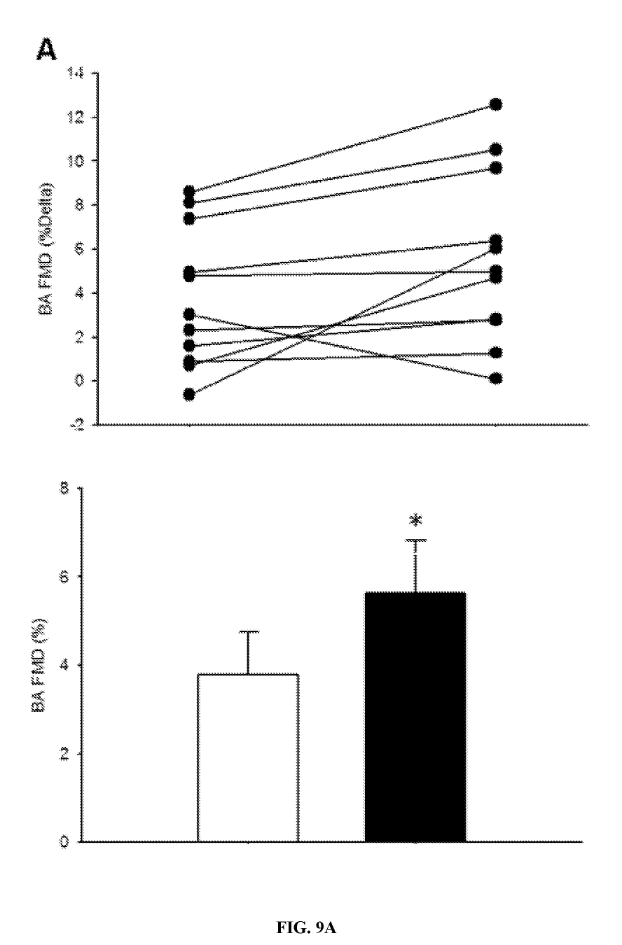

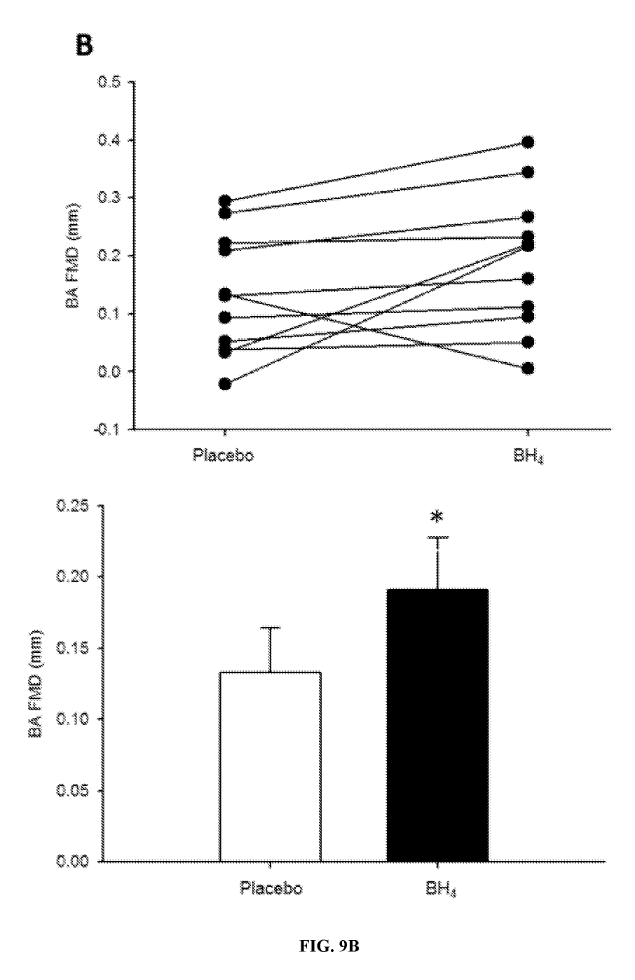

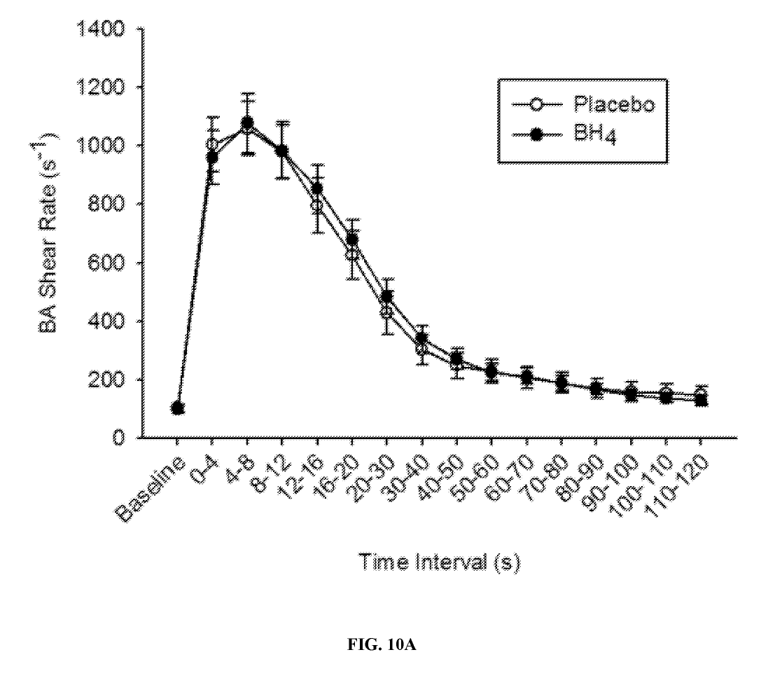

Following cuff release in the FMD protocol, both the absolute and percentage FMD were significantly higher in the subjects treated with BH4 compared to placebo (44 and 48% improvement vs. placebo, respectively; P<0.05). FIGS. 9A and 9B are graphs depicting the brachial artery (BA) flow-mediated dilation (FMD) responses between conditions, with FIG. 9A showing that the percentage FMD for individuals (upper graph) and as a group (lower graph) was ˜48% higher after BH4 compared to placebo condition. FIG. 9B shows that the absolute FMD for individuals (upper graph) and as a group (lower graph) was ˜44% higher after treatment with BH4 compared to placebo condition. *P<0.05 vs. placebo; values are Means±SEM.

Notably, in only one subject was FMD lower in the BH4 condition than in the placebo condition. In contrast, in nine of the eleven subjects, a physiologically meaningful improvement in absolute and percentage FMD of at least 20% in the BH4 condition compared to placebo was observed.

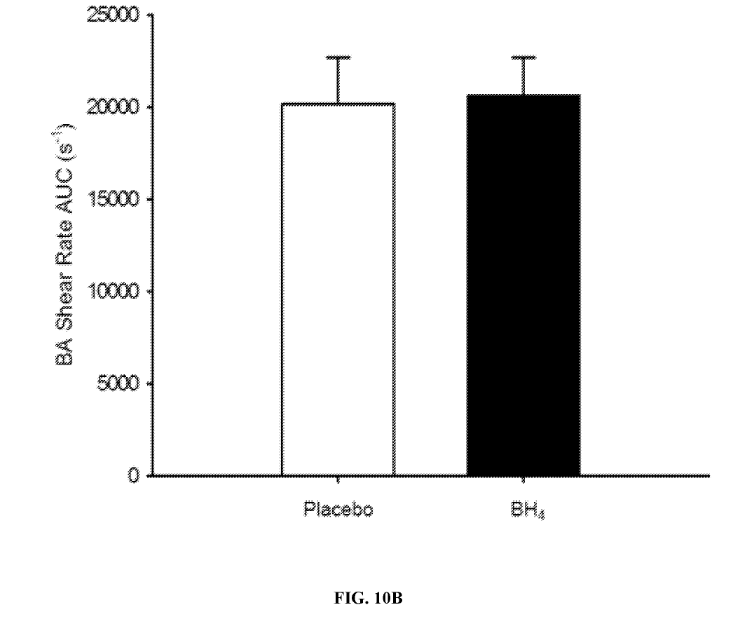

Improvements in FMD in the BH4 condition appeared to be independent of the magnitude of post-cuff release shear rate AUC leading up to peak dilation, as values were similar between conditions (P>0.05). FIG. 10A and FIG. 10B are graphs showing the hemodynamic responses to 5-min cuff occlusion. The brachial artery (BA) shear rate after 5-minute cuff occlusion is shown in FIG. 10A, as quantified by area under the curve (AUC) for shear rate across the entire 120-s post-cuff release time period. The shear rate AUC is shown in FIG. 10B, which at peak dilation was not different between placebo and BH4 conditions.

Similar hemodynamic responses following cuff release indicates that improvements in FMD in the BH4 condition is due to greater dilation to a given post-cuff release shear rate stimuli.

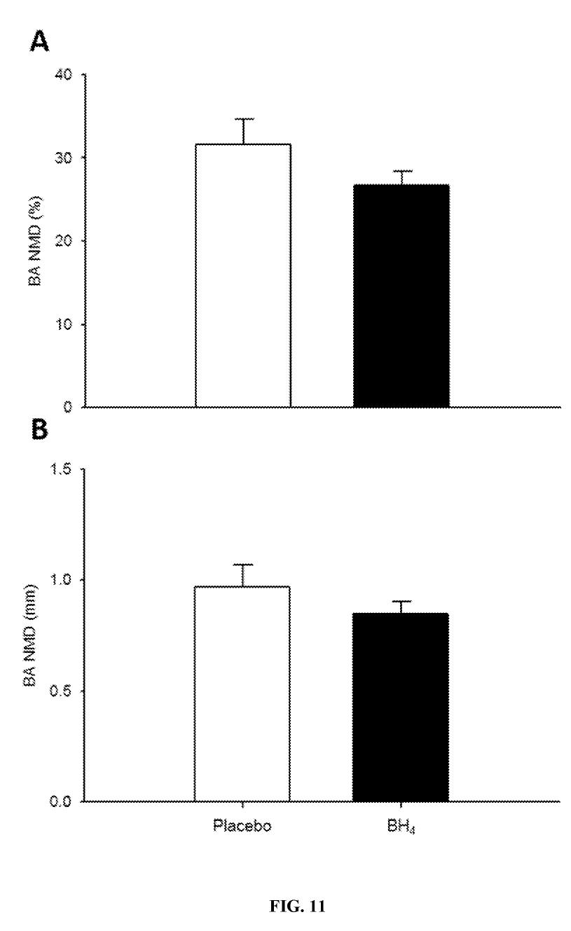

Nitroglycerin-mediated dilation (NMD) assessment was performed in the subset of SSc patients (n=4). No difference was observed between BH4 or placebo conditions in absolute or relative NMD (P>0.05). FIG. 11 shows the brachial artery (BA) NMD responses between conditions as a percentage (upper graph) and as absolute values (lower graph).

This study demonstrates that acute BH4 supplementation improved FMD in patients with SSc. Not only was a greater FMD observed following BH4 supplementation, but these improvements were achieved despite similar post-cuff release shear rate responses between conditions in view of the FMD response being at least partially NO-mediated and highly dependent on the magnitude of post-cuff release shear rate AUC. These results suggested that after BH4 supplementation, SSc patients have enhanced dilation to a given shear stimulus, which is indicative of improved NO bioavailability. Furthermore, in a subset of patients, a NMD assessment was performed and no differences between conditions were observed, indicating no effect of BH4 supplementation on smooth muscle vasoreactivity. Taken together, these results supported the view that BH4 supplementation improves FMD in patients with SSc and does so by restoring vascular endothelial function.

In this study, improvements in endothelial function were observed following acute BH4 supplementation. These data also support that acute infusion and oral BH4 supplementation can restore vascular dysfunction in various cardiovascular disease states, as well as in aging, and restore endothelial cell BH4 concentrations resulting in increased NO production and bioavailability, reduced oxidative stress, and improved endothelial function. Concomitant to the fibrosis and vasculopathy associated with SSc are low NO bioavailability and greater oxidative stress, suggesting eNOS uncoupling may be present in SSc. Although intracellular BH4 levels were not assessed in this study, these data provide evidence for eNOS recoupling as a potential mechanism by which acute BH4 supplementation improves endothelial function.

The prognosis and extent of organ involvement in SSc varies widely, but independent of disease subtypes, duration, or auto-antibody status, fibrosis and vascular disease are present in most cases of SSc. As a marker of vascular endothelial function, FMD is strongly associated with CVD and cardiovascular event risks in general. The initial studies observed impaired FMD in patients with SSc compared to age- and sex-matched healthy controls. In that study, SSc patients were subdivided into tertiles of FMD, and it was found that those in the lowest tertile (FMD <3.2%) were at 4-fold higher risk of digital ulcers than those in the highest tertile (FMD >5.4%). The second study showed that FMD values measured while in the placebo (FMD 3.2%) and BH4 (FMD 5.6%) condition corresponded to the lowest and highest tertile of FMD, respectively, as was observed in the initial study. Indeed, the magnitude of improvement in FMD from BH4 supplementation was similar to the magnitude of difference between the highest and lowest tertiles of FMD in the initial study. Therefore, BH4 supplementation while restoring endothelial function can also reduce risk of future vasculopathy complications, such as digital ulcers.

The current standard of care for treatment of SSc is the prescription of smooth muscle vasodilators (i.e., channel blockers, phosphodiesterase inhibitors, endothelin receptor antagonists, and/or prostacyclin analogs). While resting blood flow, as well as the post-cuff release shear rate, is markedly reduced in subjects with SSc as compared to healthy controls, the underlying cause of fibrosis and vasculopathy in SSc may not be exclusively blood flow-related. Patients in the second study were free of medications for 24 hours prior to testing, which likely accounts for the near three-fold reduction in resting forearm blood flow between studies. Indeed, in some patients from the second study, bedside FMD was measured while on their normal medication regimen and a two-fold reduction in resting forearm blood flow between bedside FMD and the placebo condition was observed.

While a significant decrease in MAP following BH4 supplementation was observed, the magnitude of reduction in MAP may not be physiologically relevant. Additionally, no differences in resting BA diameter, forearm blood flow, vascular resistance, or vascular conductance was observed. Therefore, the potential to treat of SSc with BH4 in combination with smooth muscle vasodilators, as per the current standard of care, would not likely increase risk associated with smooth muscle vasodilators (e.g., hypotensive shock). In a subset of patients, no differences in NMD between conditions were observed, indicating no effect of BH4 supplementation on smooth muscle vasoreactivity. Therefore, it is possible that improvement in FMD with combination treatment of BH4 and smooth muscle vasodilators targeting the endothelium and smooth muscle, respectively, could provide an additive effect.

In the second study, patients were asked to abstain from cardiovascular acting medications, primarily smooth muscle vasodilators, for 24 hours prior to testing. These drugs affect blood flow, thus, theoretically could alter FMD response; however, FMD measurements do not appear to be affected by these drugs. It is possible that 24 hours may not be enough time for these drugs to lose their efficacy. However, in a subset of patients (n=7), bedside FMD was assessed at the University of Utah Rheumatology Clinic. In these patients, resting forearm blood flow was two-fold of what was observed in the placebo condition (62±17 vs. 32±7 ml/min, respectively). Therefore, it is unlikely that there were any lingering effects of medications taken outside of the 24-hour time period prior to testing. The study was too underpowered to identify relations regarding disease duration, end-stage vasculopathy, or antibody presence on the improvements in FMD following BH4 supplementation. Not only is BH4 an essential cofactor for eNOS, but is also an antioxidant. Therefore, the possibility that reductions in blood oxidative stress may have been responsible for increases in FMD observed in the present study cannot be excluded. However, it should be noted that acute oral antioxidant supplementation does not appear to improve FMD.

These studies have shown that BA FMD is improved with BH4 supplementation in patients with SSc. These improvements were achieved despite similar post-cuff release shear stimuli between conditions and indicated that BH4 supplementation improves FMD in patients with SSc by restoring vascular endothelial function.

### Example 3

Systemic sclerosis (SSc) is a rare systemic auto-immune disease characterized by fibrosis of the skin and internal organs, as well as a reduced exercise capacity. It has been demonstrated that patients with SSc have peripheral arterial vascular dysfunction, but whether that accounts for reduced exercise capacity in SSc has never been investigated. Here, 15 age- and sex-matched healthy controls (10 women:5 men; age 56±5 yrs) and 10 patients with SSc (7 women, 3 men; age 61±4 yrs) were recruited to perform intermittent static progressive handgrip exercise (1 Hz, 3 min) at intensities corresponding to 15, 30, and 45% of maximal voluntary contraction (MVC). Cardiovascular measurements were determined at baseline and the final minute of each workload. Healthy controls and patients with SSc were similar in body stature, handgrip MVC, and MAP (P>0.05), however, resting forearm blood flow and brachial artery lumen diameter were significantly lower and forearm vascular resistance significantly higher in patients with SSc compared to healthy controls (blood flow: 22±4 vs. 42±5 ml/min; lumen diameter: 3.06±0.16 vs. 3.72±0.17 mm; vascular resistance: 4.7±0.6 vs. 2.6±0.3 U, P<0.05).

Despite similar BA vasodilation and increases in MAP, forearm blood flow and vascular conductance were ˜32-39% lower at each handgrip workload in patients with SSc compared to healthy controls (P<0.05). Although there were no differences in BA vasodilation during exercise (P>0.05), the relationship between the change in BA diameter and the change in shear rate exhibited a significant downward shift in patients with SSc (P<0.05). In addition, vascular dysfunction was associated with elevated blood markers of oxidative stress and attenuated endogenous antioxidant activity in patients with SSc (P<0.05). Together, these findings indicate that the peripheral vascular hemodynamic response to progressive handgrip exercise is impaired in patients with SSc and may be partly responsible for the attenuated exercise capacity in SSc. While there are several potential mechanisms that may be responsible for impaired exercise-induced forearm blood flow, increased peripheral vasoconstriction, endothelial dysfunction, and oxidative stress appear to play a role.

Systemic sclerosis (Scleroderma; SSc) is a rare auto-immune disease that results in fibrosis of the skin and internal organs, and a median survival of ˜11 yrs post diagnosis. The cause of SSc is unknown, and there is no cure or effective treatment available, as heterogeneity in the extent of organ involvement varies considerably between patients. Despite the heterogeneous nature of SSc, systemic vascular dysfunction is present in nearly all patients. Furthermore, peripheral perfusion abnormalities, such as alterations in the vasoconstrictor and vasodilator signaling in digital resistance arteries, are a major diagnostic criteria for SSc and occur in an overwhelming majority of patients.

In addition to vascular dysfunction, patients with SSc also have an attenuated exercise capacity. Although several studies have shown that attenuated exercise capacity in SSc is due to central mechanisms (i.e., cardiopulmonary abnormalities), others have found that exercise capacity is attenuated independent of central hemodynamic impairments. Considering the prevalence of peripheral perfusion abnormalities in SSc, it is likely that peripheral mechanisms plays a role in the attenuated exercise capacity in SSc. Surprisingly, there are no studies to date that have examined peripheral factors (i.e., limb blood flow) that might limit exercise capacity in SSc. Progressive handgrip exercise is a common exercise model that incorporates a small amount of muscle mass, and requires a small fraction of maximal cardiac output, negating the impact of central hemodynamic factors. Therefore, using this model, an investigation of peripheral hemodynamic factors that might limit exercise capacity in patients with SSc was performed.

Subjects. Ten patients with SSc were recruited from the University of Utah SSc Clinic. Patients had either a diagnosis of SSc as accepted by the American College of Rheumatology or early SSc as described by Leroy and Medsger. Clinical features of the SSc patients were recorded and displayed in Table 3. Fifteen age- and sex-matched healthy controls were recruited from the general population. Healthy control subjects did not have any evidence of vascular disease or chronic medical conditions and were not on any medications that would impact vascular function. All procedures were approved by the institutional review board, which serves as the ethics committee, of the University of Utah and Salt Lake City VAMC. The nature, benefits, and risks of the study were explained to the subjects, and their written informed consent was obtained before participation. Patients were excluded if they had used cardiovascular acting medications, tobacco, alcohol, and/or caffeine within 12 h of testing. Seventy percent (70%) of patients with SSc were taking calcium-channel blockers. These, as well as other cardiovascular acting medications, were discontinued 12 hours prior to study visit. In premenopausal women, measurements were performed during the early follicular phase of the menstrual cycle.

Subject Characteristics. Body mass index (BMI) was calculated from body mass and height. For patients with SSc, clinical features were measured and recorded, including disease duration, medical history (digital ulcers [DU], pulmonary arterial hypertension [PAH], and scleroderma renal crisis [SRC]), and SSc-antibodies status.

Progressive Handgrip Exercise. Subjects reported to the laboratory fasted for at least 5 hours then performed static intermittent handgrip exercise. Subjects exercised at 15, 30, and 45% of the maximal volitional contraction (MVC). Each exercise stage was performed for 3 min with a 2-min break allotted between each workload.

Measurements. Heart rate (HR) was monitored from a standard three-lead ECG. Mean arterial blood pressure (MAP) was measured on the contralateral arm by auscultation of the brachial artery (Tango+, SunTech, Morrisville, N.C.). Simultaneous measurements of brachial artery blood velocity and vessel diameter were performed. Shear rate was calculated according to the equation: shear rate (s⁻¹)=(blood velocity×8)/vessel diameter. Forearm blood flow was calculated as per the equation: blood flow (mL/min) =(blood velocity×π)×(vessel diameter/2)²×(60). Forearm vascular conductance was calculated according to the equation: blood flow/MAP. Forearm vascular resistance was calculated according the equation: MAP/blood flow. All measurements were performed in the final minute of handgrip exercise.

Oxidative stress, antioxidant capacity, and inflammation assays. Blood samples were obtained from the antecubital vein in healthy controls (n=6) and patients with SSc (n=6). Serum and plasma samples were stored at −80° C. until analysis. Lipid peroxidation, a marker of oxidative stress, was assessed by quantifying plasma malondialdehyde (MDA) levels (Oxis Research/Percipio Bioscience, Foster City, Calif.). Protein carbonyl levels were measured by a protein carbonyl ELISA assay (Northwest Life Science Specialties, LLC Vancouver, Wash.). Endogenous antioxidant activity, assessed by superoxide dismutase (SOD) and catalase (CAT) activity, was assayed in the plasma (Cayman Chemical Company, Ann Arbor, Mich.). Antioxidant capacity was assessed by determining the ferric reducing ability of plasma (FRAP), using the method described by Benzie and Strain. Systemic inflammation was assessed by determining TNF-α and CRP assayed in the serum (R&D Systems, Minneapolis, Minn.).

Statistics. Statistics were performed using SPSS software (IBM, Chicago, Ill.). Unpaired t-tests were used to compare differences in subject characteristics, cardiovascular variables at rest, and slope of the relationship of brachial artery vasodilation to shear rate between SSc and healthy patients. A two-way repeated-measures ANOVA was used to evaluate differences between healthy and SSc patients during exercise, and a least significant difference unpaired t-test identified means that were significantly different. Statistical significance was set at P<0.05 for all analyses. Data are presented as Means±SEM.

Subject characteristics. Patients with SSc and healthy controls were well matched for age, sex, body stature, and handgrip MVC (P>0.05; Table 3). Among patients with SSc, the duration of SSc ranged from 1-14 years (mean 5.3 years).

TABLE 3
Subject characteristics and blood chemistries.

| | Healthy | Scleroderma |
|---|---|---|
| Women:men | 10:5 | 7:3 |
| Age, yrs | 56 ± 5 | 61 ± 4 |
| Height, cm | 169 ± 3 | 170 ± 3 |
| Weight, kg | 68.6 ± 3.0 | 69.4 ± 3 |
| BMI, kg/m² | 24.0 ± 0.7 | 24.0 ± 0.9 |
| MVC, kg | 19.1 ± 1.1 | 17.3 ± 2.0 |
| SSc duration, yrs | — | 5 ± 1 |
| Medications, n (%) | | |
| Calcium channel blockers | — | 7 (70) |
| Endothelin receptor antagonists | — | 0 (0) |
| Phosphodiesterase inhibitors | — | 0 (0) |
| Immunosuppression | — | 4 (40) |
| Medical history, n (%) | | |
| Digital ulcers | — | 6 (60) |
| Scleroderma renal crisis | — | 1 (10) |
| Pulmonary arterial hypertension | — | 1 (10) |
| Antibody presence, n (%) | | |
| Antinuclear antibody | — | 10 (100) |
| Centromere | — | 5 (50) |
| RNA polymerase III | — | 1 (10) |
| SCL70 | — | 2 (20) |
| Fibrillin | — | 2 (20) |
| Th/To | — | 0 (0) |
| RNP | — | 1 (10) |

Values are Means ± SEM.

Oxidative stress, antioxidant capacity, and inflammation. Lipid peroxidation, as measured by plasma MDA levels, was significantly higher in patients with SSc compared to healthy controls (P<0.05; Table 4).

TABLE 4
Blood oxidative stress, antioxidant status, and inflammatory markers.

| | Healthy | Scleroderma |
|---|---|---|
| MDA, μM | 1.27 ± 0.26 | 2.8 ± 0.1* |
| Protein carbonyl, nM/mg | 0.14 ± 0.01 | 0.16 ± 0.01^{P-0.06} |
| CAT, nM/min/mL | 117 ± 17 | 76 ± 8* |
| FRAP, nM/L | 1.6 ± 0.1 | 1.7 ± 0.1 |
| SOD, U/mL | 8.9 ± 0.6 | 10.4 ± 0.6 |
| TNF-α, pg/mL | 0.7 ± 0.1 | 1.1 ± 0.2^{P-0.08} |
| CRP, mg/L | 1.62 ± 0.4 | 2.4 ± 0.5^{P-0.011} |

Values are Means ± SEM.
*P < 0.05 vs. healthy subjects.

Likewise, there was a trend for higher plasma protein carbonyl levels in patients with SSc (P=0.06). Additionally, endogenous antioxidant activity, as measured by plasma CAT levels, was significantly lower in patients with SSc (P<0.05). There were no differences in plasma FRAP or SOD levels between groups (P>0.05). There was a trend for higher serum TNF-α and CRP levels in patients with SSc (P=0.08-0.11), indicating elevated inflammation in SSc.

Cardiovascular variables at rest. Selected cardiovascular variables of patients with SSc and healthy controls at rest are presented in Table 5.

TABLE 5
Cardiovascular variables at rest.

| | Healthy | Scleroderma |
|---|---|---|
| Heart rate, bpm | 54 ± 2 | 65 ± 3* |
| Mean arterial pressure, mmHg | 89 ± 2 | 85 ± 2 |
| Lumen diameter, mm | 3.72 ± 0.17 | 3.06 ± 0.16* |
| Wall thickness, mm | 0.29 ± 0.03 | 0.37 ± 0.02* |
| Wall-to-lumen ratio, mm | 0.08 ± 0.02 | 0.12 ± 0.02* |
| Blood velocity, cm/sec | 6.4 ± 0.7 | 4.7 ± 0.4* |
| Shear rate, s⁻¹ | 72 ± 9 | 62 ± 5 |
| Forearm blood flow, ml/min | 42 ± 5 | 22 ± 4* |
| Forearm vascular conductance, U | 0.5 ± 0.1 | 0.3 ± 0.0* |
| Forearm vascular resistance, U | 2.6 ± 0.3 | 4.7 ± 0.6* |

Values are Means ± SEM.
*P < 0.05 vs. healthy subjects.

In SSc patients, HR was elevated at rest compared to healthy controls (P<0.05), but there were no differences in MAP between groups (P>0.05). Although resting brachial artery lumen diameter was significantly smaller in SSc compared to healthy controls (P<0.05), patients with SSc had greater wall thickness and wall-to-lumen ratio than healthy controls (P<0.05). Resting brachial artery blood velocity, forearm blood flow, and forearm vascular conductance were all significantly lower in SSc compared to healthy controls (P<0.05), while forearm vascular resistance was ˜80% higher in patients with SSc (P<0.05).

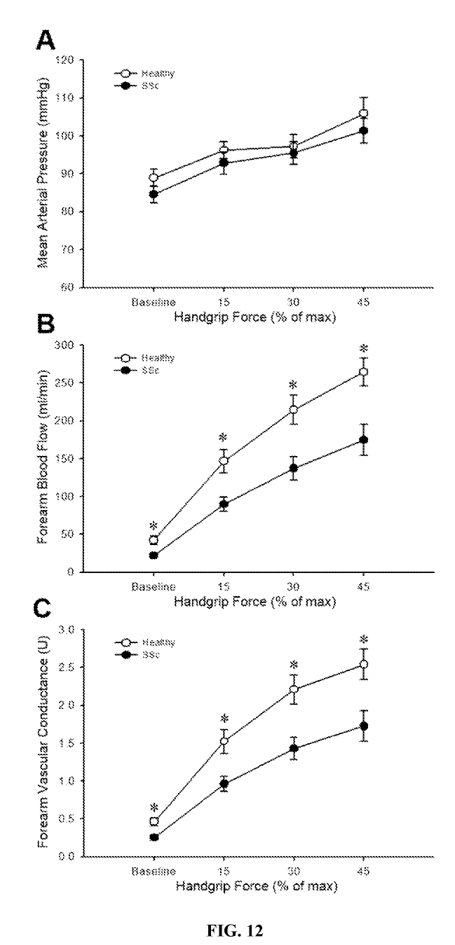

Cardiovascular variables during handgrip exercise. Despite similar brachial artery vasodilation and exercise pressor response, forearm blood flow was ˜34-39% lower at each handgrip workload in patients with SSc compared to healthy controls (FIG. 12; P<0.05).

In FIG. 12, graphs show the mean arterial pressure (FIG. 12A), forearm blood flow (FIG. 12B), and forearm vascular conductance (FIG. 12C) during progressive handgrip exercise in the healthy controls (white circles) and patients with systemic sclerosis (SSc; black circles). *P<0.05, significant difference between control and SSc trials. All data are Means±SEM.

Consequently, there was a ˜32-37% reduction in forearm vascular conductance at each workload in SSc, as shown in the graphs. Similar to at rest, at all exercise workloads lumen diameter was significantly smaller in SSc compared to healthy (Table 6; P<0.05).

TABLE 6
Cardiovascular variables during exercise.

| | Exercise Intensity | | |
|---|---|---|---|
| Relative, % MVC | 15% | 30% | 45% |
| Healthy | | | |
| Heart rate, bpm | 63 ± 2 | 62 ± 3 | 66 ± 3 |
| Lumen diameter, mm | 3.86 ± 0.16 | 4.02 ± 0.15 | 4.18 ± 0.14 |
| Vasodilation, % | 4.1 ± 0.9 | 8.8 ± 1.7 | 13.3 ± 2.0 |
| Blood velocity, cm/sec | 20.5 ± 1.4 | 28.0 ± 1.7 | 32.4 ± 1.7 |
| Shear rate, s⁻¹ | 219 ± 17 | 285 ± 20 | 319 ± 24 |
| SSc | | | |
| Heart rate, bpm | 69 ± 3* | 71 ± 3* | 73 ± 3^{P-0.07} |
| Lumen diameter, mm | 3.22 ± 0.15* | 3.31 ± 0.16* | 3.46 ± 0.17* |
| Vasodilation, % | 5.5 ± 1.7 | 8.4 ± 2.5 | 13.4 ± 2.5 |
| Blood velocity, cm/sec | 18.8 ± 2.0 | 26.9 ± 2.4 | 30.9 ± 2.5 |
| Shear rate, s⁻¹ | 242 ± 32 | 337 ± 39 | 369 ± 38 |

Values are Means ± SEM.
*P < 0.05 vs. healthy subjects.

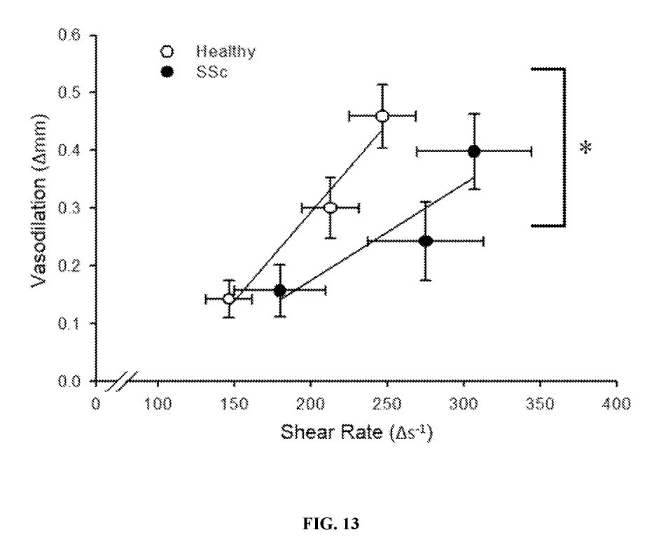

There were no differences in brachial artery vasodilation or blood velocity during exercise between SSc patients as compared to control patients (P>0.05). Although the shear rate tended to be higher during exercise in patients with SSc, these differences did not reach statistical significance (P>0.05). However, the relationship between A brachial artery diameter and A shear rate exhibited a significant downward shift in patients with SSc, as seen in the graph of FIG. 13.

FIG. 13 shows the relationship between changes in brachial artery shear rate and the associated change in vasodilation during each stage (15, 30, and 45% MVC) of handgrip exercise in healthy controls (white circles) and patients with systemic sclerosis (SSc; black circles). A significant reduction in slope was evident in patients with SSc compared with healthy controls. *P<0.05, significant difference between slope of healthy controls and SSc. All data are Means±SEM.

There are several novel observations from this study. First, an impairment in exercise-induced forearm blood flow to progressive handgrip exercise in patients with SSc has been documented. Second, impairments in exercise-induced forearm blood flow appear to be mediated by peripheral vasoconstriction of resistance arteries, as evidenced by increased vascular resistance at rest and an impaired ability to increase vascular conductance during exercise. Third, in addition to a dysfunctional resistance arterial vasculature, an impaired ability of the brachial artery to vasodilate in response to increases in shear rate was observed. This indicates that vascular dysfunction in SSc is also present in the larger conduit arteries, as well. Lastly, elevated blood markers of oxidative stress and attenuated antioxidant capacity were associated with vascular dysfunction in SSc. Together, these findings indicate that the peripheral vascular hemodynamic response to progressive handgrip exercise is impaired in patients with SSc and may contribute to the attenuated exercise capacity in SSc patients. While there are several potential mechanisms that may be responsible for this, increased oxidative stress appears to play a role.

In patients with SSc, an impairment in exercise-induced forearm blood flow during progressive handgrip exercise was observed. This exercise model requires a small fraction of maximal cardiac output. Thus, impairments in exercise-induced blood flow may be due to a dysfunctional peripheral vasculature in SSc. Although previous studies have implicated central hemodynamic impairments (i.e., cardiopulmonary abnormalities), it appears that peripheral mechanisms may play a major role in the attenuated exercise capacity in SSc. Several studies, including the present study, have demonstrated peripheral vascular dysfunction in patients with SSc at rest. While the effects of cardiopulmonary abnormalities and other central hemodynamic impairments on exercise capacity are undeniable, clearly peripheral hemodynamic factors should not be overlooked. Moreover, many of the pathophysiological effects of SSc, such as perfusion abnormalities and vascular dysfunction, affect central and peripheral hemodynamics alike.

In addition to impaired exercise-induced forearm blood flow, SSc patients also exhibited an increased vascular resistance at rest and an impaired ability to increase vascular conductance during exercise. Increases in vascular conductance during exercise reflect the ability of resistance arteries and microvasculature downstream of the brachial artery to vasodilate. The inability to do so indicates peripheral vasoconstriction impairs the ability to appropriately increase blood flow during exercise. In support of this notion, earlier studies by the inventors indicated that the hyperemic response to an ischemic challenge (i.e., brachial flow-mediated dilation) was nearly half that of healthy controls. Given that patients with SSc are known to have dysfunctional vasoconstrictor and vasodilator signaling in digital resistance arteries, it is likely that peripheral vasoconstriction limits the ability to appropriately increase vascular conductance, thereby impairing exercise-induced blood flow in SSc.

During progressive handgrip exercise, an increase in blood flow to the exercising musculature induces vasodilation of the brachial artery. At first glance, exercise-induced brachial artery vasodilation appeared to be preserved in patients with SSc. However, vasodilation to a given increase shear rate was lower in SSc, which may indicate endothelial dysfunction in the brachial artery, as vasodilation to increases in shear rate during progressive handgrip exercise has been shown to be mediated by nitric oxide. To further support the notion that endothelial dysfunction is present in the brachial artery of SSc patients, previous studies by the inventors observed impaired brachial artery flow-mediated dilation when normalized to shear rate in SSc patients, but also point to differences in the structural characteristics of the artery. Similar to the previous studies, patients with SSc had a smaller brachial artery lumen diameter.

Considering the inverse relationship of flow-mediated dilation to resting lumen diameter, for vasodilation to be considered ‘healthy’ in SSc patients, theoretically, it would need to be greater than that of a larger diameter artery, not equal. Additionally, increased brachial artery wall thickness, as observed in SSc patients in this study, is indicative of increased vascular tone and represents another barrier to vasodilation, as wall thickness is inversely related to flow-mediated dilation. Together, these findings indicate that despite a seemingly preserved vasodilation response in SSc patients, structural and functional abnormalities of the brachial artery, namely endothelial dysfunction, may limit its true vasodilatory capacity.

In the present study, vascular dysfunction was accompanied by elevated blood markers of oxidative stress and attenuated antioxidant capacity, as well as a trend for elevated inflammation. An unbalanced redox state, in favor of oxidative stress, is known to contribute to vascular dysfunction during handgrip exercise. While the functional consequences of oxidative stress are widespread, the vascular endothelium is particularly vulnerable to oxidative damage from reactive oxygen species. Indeed, elevations in oxidative stress have been implicated for abnormal nitric oxide metabolism and up-regulation of vasoconstrictors endothelin-1 and asymmetric dimethylarginine in patients with SSc, and may be responsible for vascular dysfunction and structural abnormalities observed in this study. Although it is beyond the scope of this study to determine mechanisms of oxidative stress-induced vascular dysfunction and remodeling in SSc, it is likely that oxidative stress plays a major role in SSc-related vascular dysfunction at rest and during exercise.

This study indicates that treatment or supplementation with BH4 can be used for any disease which results in a patient exhibiting a reduced exercise capacity due to a decrease in their peripheral blood flow, including systemic sclerosis, chronic heart failure, peripheral vascular disease and diabetes.

Patients in the study had abstained from cardiovascular-acting medications for at least 12 hours prior to the testing visit, however, it cannot be determined if medications taken outside of the 12 hour window had an impact on vascular function. Nevertheless, resting blood flow was lower in this study compared to previous work by the inventors, in which patients maintained medications. This suggests that any effect of cardiovascular-acting medications was negligible. Although NO has been shown to be a significant mediator of exercise-induced blood flow during handgrip exercise, no measurements were made here in the presence and absence of intra-arterial L-NMMA infusion. Thus, it cannot be confirmed that NO-mediated endothelial dysfunction limits exercise-induced blood flow in SSc.

In summary, impairments in exercise-induced forearm blood flow to progressive handgrip exercise have been documented, which appear to be mediated by peripheral vasoconstriction of resistance arteries, as well as an impaired ability of the brachial artery to vasodilate in response to increases in shear rate. Additionally, peripheral vascular dysfunction in SSc was associated with elevated blood markers of oxidative stress and attenuated antioxidant capacity. Together, these findings indicate that in addition to central hemodynamic impairments, reduced exercise capacity in SSc patients may also be due to peripheral mechanisms, as well. These findings suggest that smooth muscle vasodilatory and endothelium-targeted drugs, independently or in combination, may be able to ameliorate impairments in exercise-induced blood flow, vascular conductance, and vascular dysfunction, in general, in patients with SSc.

### REFERENCES

Each of the following citations is fully incorporated herein by reference in its entirety.
1. Mayes, M. D., et al., Prevalence, incidence, survival, and disease characteristics of systemic sclerosis in a large US population. Arthritis Rheum, 2003. 48(8): p. 2246-55.
2. Kuwana, M., et al., Defective vasculogenesis in systemic sclerosis. Lancet, 2004. 364(9434): p. 603-10.
3. Frech, T. M., et al., Vascular leak is a central feature in the pathogenesis of systemic sclerosis. J Rheumatol, 2012. 39(7): p. 1385-91.
4. Sgonc, R., et al., Endothelial cell apoptosis is a primary pathogenetic event underlying skin lesions in avian and human scleroderma. J Clin Invest, 1996. 98(3): p. 785-92.
5. Frech, T., et al., Systemic sclerosis induces pronounced peripheral vascular dysfunction characterized by blunted peripheral vasoreactivity and endothelial dysfunction. Clin Rheumatol, 2015. 34(5): p. 905-13.
6. Cosentino, F. and T. F. Luscher, Tetrahydrobiopterin and endothelial function. Eur Heart J, 1998. 19 Suppl G: p. G3-8.
7. Moncada, S., R. M. Palmer, and E. A. Higgs, Nitric oxide: physiology, pathophysiology, and pharmacology. Pharmacol Rev, 1991. 43(2): p. 109-42.
8. Wever, R. M., et al., Tetrahydrobiopterin regulates superoxide and nitric oxide generation by recombinant endothelial nitric oxide synthase. Biochem Biophys Res Commun, 1997. 237(2): p. 340-4.
9. Schmidt, T. S. and N. J. Alp, Mechanisms for the role of tetrahydrobiopterin in endothelial function and vascular disease. Clin Sci (Lond), 2007. 113(2): p. 47-63.
10. Shinozaki, K., et al., Oral administration of tetrahydrobiopterin prevents endothelial dysfunction and vascular oxidative stress in the aortas of insulin-resistant rats. Circ Res, 2000. 87(7): p. 566-73.
11. Maier, W., et al., Tetrahydrobiopterin improves endothelial function in patients with coronary artery disease. J Cardiovasc Pharmacol, 2000. 35(2): p. 173-8.
12. Eskurza, I., et al., Tetrahydrobiopterin augments endothelium-dependent dilatation in sedentary but not in habitually exercising older adults. J Physiol, 2005. 568(Pt 3): p. 1057-65.
13. Heitzer, T., et al., Tetrahydrobiopterin improves endothelium-dependent vasodilation in chronic smokers: evidence for a dysfunctional nitric oxide synthase. Circ Res, 2000. 86(2): p. E36-41.
14. Heitzer, T., et al., Tetrahydrobiopterin improves endothelium-dependent vasodilation by increasing nitric oxide activity in patients with Type II diabetes mellitus. Diabetologia, 2000. 43(11): p. 1435-8.
15. Preliminary criteria for the classification of systemic sclerosis (scleroderma). Subcommittee for scleroderma criteria of the American Rheumatism Association Diagnostic and Therapeutic Criteria Committee. Arthritis Rheum, 1980. 23(5): p. 581-90.
16. LeRoy, E. C. and T. A. Medsger, Jr., Criteria for the classification of early systemic sclerosis. J Rheumatol, 2001. 28(7): p. 1573-6.
17. Machin, D. R., et al., Hypotensive effects of solitary addition of conventional nonfat dairy products to the routine diet: a randomized controlled trial. Am J Clin Nutr, 2014. 100(1): p. 80-7.
18. Harris, R. A., et al., Ultrasound assessment of flow-mediated dilation. Hypertension, 2010. 55(5): p. 1075-85.
19. Wray, D. W., et al., Does brachial artery flow-mediated vasodilation provide a bioassay for NO? Hypertension, 2013. 62(2): p. 345-51.
20. Pyke, K. E. and M. E. Tschakovsky, Peak vs. total reactive hyperemia: which determines the magnitude of flow-mediated dilation? J Appl Physiol (1985), 2007. 102(4): p. 1510-9.
21. Ogawa, F., et al., Serum levels of 8-isoprostane, a marker of oxidative stress, are elevated in patients with systemic sclerosis. Rheumatology (Oxford), 2006. 45(7): p. 815-8.
22. Celermajer, D. S., et al., Non-invasive detection of endothelial dysfunction in children and adults at risk of atherosclerosis. Lancet, 1992. 340(8828): p. 1111-5.
23. Green, D. J., et al., Flow-mediated dilation and cardiovascular event prediction: does nitric oxide matter? Hypertension, 2011. 57(3): p. 363-9.
24. Wigley, F. M., et al., The post-occlusive hyperemic response in patients with systemic sclerosis. Arthritis Rheum, 1990. 33(11): p. 1620-5.
25. Harris, R. A., et al., The effect of oral antioxidants on brachial artery flow-mediated dilation following 5 and 10 min of ischemia. Eur J Appl Physiol, 2009. 107(4): p. 445-53.
26. Nietert, P. J., M. D. Silverstein, and R. M. Silver, Hospital admissions, length of stay, charges, and in-hospital death among patients with systemic sclerosis. J Rheumatol, 2001. 28(9): p. 2031-7.
27. Blom-Bülow, B., B. Jonson, and K. Brauer. Factors limiting exercise performance in progressive systemic sclerosis. in Semin Arthritis Rheum. 1983. Elsevier.
28. Kovacs, G., et al., Borderline pulmonary arterial pressure is associated with decreased exercise capacity in scleroderma. American journal of respiratory and critical care medicine, 2009. 180(9): p. 881-886.
29. Callejas-Rubio, J. L., et al., Prevalence of exercise pulmonary arterial hypertension in scleroderma. J Rheumatol, 2008. 35(9): p. 1812-1816.
30. Alkotob, M. L., et al., Reduced exercise capacity and stress-induced pulmonary hypertension in patients with scleroderma. CHEST Journal, 2006. 130(1): p. 176-181.
31. D'Alto, M., et al., Inappropriate exercise-induced increase in pulmonary artery pressure in patients with systemic sclerosis. Heart, 2011. 97(2): p. 112-117.
32. de Oliveira, N. C., et al., Reduced exercise capacity in systemic sclerosis patients without pulmonary involvement. Scand J Rheumatol, 2007. 36(6): p. 458-61.
33. Wray, D. W., et al., Progressive handgrip exercise: evidence of nitric oxide-dependent vasodilation and blood flow regulation in humans. Am J Physiol Heart Circ Physiol, 2011. 300(3): p. H1101-7.
34. Wheeler, C. R., et al., Automated assays for superoxide dismutase, catalase, glutathione peroxidase, and glutathione reductase activity. Anal Biochem, 1990. 184(2): p. 193-9.
35. Benzie, I. F. and J. J. Strain, The ferric reducing ability of plasma (FRAP) as a measure of “antioxidant power”: the FRAP assay. Anal Biochem, 1996. 239(1): p. 70-6.
36. Chen, X., et al., TNF-alpha, a potent lipid metabolism regulator. Cell Biochem Funct, 2009. 27(7): p. 407-16.
37. Goodfield, M. J., A. Hume, and N. R. Rowell, The effect of simple warming procedures on finger blood flow in systemic sclerosis. Br J Dermatol, 1988. 118(5): p. 661-8.
38. Katz, S. D., et al., Training improves endothelium-dependent vasodilation in resistance vessels of patients with heart failure. J Appl Physiol (1985), 1997. 82(5): p. 1488-92.
39. Donato, A. J., et al., Exercise-induced brachial artery vasodilation: effects of antioxidants and exercise training in elderly men. Am J Physiol Heart Circ Physiol, 2010. 298(2): p. H671-8.
40. Trinity, J. D., et al., Ascorbic Acid Improves Brachial Artery Vasodilation during Progressive Handgrip Exercise in the Elderly through a Nitric Oxide Mediated Mechanism. Am J Physiol Heart Circ Physiol, 2016: p. ajpheart 00817 2015.
41. Trinity, J. D., et al., Contribution of nitric oxide to brachial artery vasodilation during progressive handgrip exercise in the elderly. Am J Physiol Regul Integr Comp Physiol, 2013. 305(8): p. R893-9.
42. Thijssen, D. H., et al., Impact of wall thickness on conduit artery function in humans: is there a “Folkow” effect? Atherosclerosis, 2011. 217(2): p. 415-9.
43. Cai, H. and D. G. Harrison, Endothelial dysfunction in cardiovascular diseases: the role of oxidant stress. Circ Res, 2000. 87(10): p. 840-4.
44. Dooley, A., et al., Abnormal nitric oxide metabolism in systemic sclerosis: increased levels of nitrated proteins and asymmetric dimethylarginine. Rheumatology (Oxford), 2006. 45(6): p. 676-84.
45. Pu, Q., et al., Endothelin antagonism on aldosterone-induced oxidative stress and vascular remodeling. Hypertension, 2003. 42(1): p. 49-55.
46. Shimizu, K., et al., Increased serum levels of nitrotyrosine, a marker for peroxynitrite production, in systemic sclerosis. Clin Exp Rheumatol, 2007. 25(2): p. 281-6.

Various features and advantages of the invention are set forth in the following claims.

What is claimed is:

1. A method of treating systemic sclerosis, comprising administering an effective amount of tetrahydrobiopterin to a patient in need thereof.

2. The method of claim 1, wherein the patient's mean arterial blood pressure is not substantially affected by the administration.

3. The method of claim 1, wherein the patient's brachial artery shear rate is not substantially affected by the administration.

4. The method of claim 1, further comprising administration of at least one smooth muscle dilating drug.

5. The method of claim 1, further comprising administration of at least one drug which causes constriction of the pulmonary blood vessels.

6. The method of claim 1, wherein the tetrahydrobiopterin is administered orally.

7. The method of claim 1, wherein the tetrahydrobiopterin is administered once a day.

8. The method of claim 1, wherein the tetrahydrobiopterin is administered more than once a day.

9. The method of claim 1, wherein the effective amount of tetrahydrobiopterin is a dosage between about 0.5 mg/kg and about 50 mg/kg.

10. The method of claim 1, wherein the effective amount of tetrahydrobiopterin is a dosage between about 1 mg/kg and about 15 mg/kg.

11. The method of claim 1, wherein administering the tetrahydrobiopterin results in the patient having an average brachial artery dilation to flow of at least about 0.20 mm.

12. The method of claim 1, wherein administering the tetrahydrobiopterin results in the patient having an increase in their brachial artery dilation to flow of at least about 30%.

13. The method of claim 1, wherein administering the tetrahydrobiopterin results in the patient having an increase in the average brachial artery flow mediated diameter of at least about 0.05 mm as compared to the average diameter prior to treatment.

14. The method of claim 1, wherein administering the tetrahydrobiopterin results in the patient having an increase in the average brachial artery flow mediated diameter of at least about 2% as compared to the average diameter prior to treatment.

15. The method of claim 1, wherein administering the tetrahydrobiopterin results in a decrease in the risk of getting a digital ulcer as compared to a patient administered a placebo.

16. The method of claim 15, wherein administering the tetrahydrobiopterin results in a decrease in the risk of getting a digital ulcer from about 45% to about 15% as compared to a patient administered a placebo.

17. A method of treating a disease which is associated with a reduced exercise capacity due to a decrease in a patient's peripheral blood flow, comprising administering an effective amount of tetrahydrobiopterin to a patient in need thereof.

18. The method of claim 17, wherein the patient's mean arterial blood pressure is not substantially affected by the administration.

19. The method of claim 17, wherein the patient's brachial artery shear rate is not substantially affected by the administration.

20. The method of claim 17, further comprising administration of at least one smooth muscle dilating drug.

21. The method of claim 17, further comprising administration of at least one drug which causes constriction of the pulmonary blood vessels.

22. The method of claim 17, wherein the tetrahydrobiopterin is administered orally.

23. The method of claim 17, wherein the tetrahydrobiopterin is administered once a day.

24. The method of claim 17, wherein the tetrahydrobiopterin is administered more than once a day.

25. The method of claim 17, wherein the effective amount of tetrahydrobiopterin is a dosage between about 0.5 mg/kg and about 50 mg/kg.

26. The method of claim 17, wherein the effective amount of tetrahydrobiopterin is a dosage between about 1 mg/kg and about 15 mg/kg.

27. The method of claim 17, wherein administering the tetrahydrobiopterin results in the patient having an increase in their average forearm blood flow of at least about 20% as compared to the average forearm blood flow prior to treatment.

28. The method of claim 17, wherein administering the tetrahydrobiopterin results in the patient having an increase in their average forearm blood flow of at least about 30 mL/min as compared to the average forearm blood flow prior to treatment.

29. The method of claim 17, wherein the disease is at least one of chronic heart failure, peripheral vascular disease, diabetes or systemic sclerosis.
